(19) United States
(12) Patent Application Publication
(10) Pub. No.: US 2026/0232773 A1
(43) Pub. Date: Aug. 13, 2026

(54) COMPOSITIONS AND METHODS FOR INFUSION OF NUTS, SEEDS, LEGUMES, GRAINS AND COFFEE HUSKS

(71) Applicant: Eterno Wellvations LLC, Miami Beach, FL (US)

(72) Inventors: Ted Kelly Oliver, Miami Beach, FL (US); Michael Thomas Taraska, Miami Beach, FL (US)

(21) Appl. No.: 19/637,945

(22) Filed: Apr. 2, 2026

Related U.S. Application Data
(63) Continuation of application No. PCT/US2024/053264, filed on Oct. 28, 2024.
(60) Provisional application No. 63/594,125, filed on Oct. 30, 2023.

(51) Int. Cl. A61K 38/39; A23F 5/02; A23F 5/14; A23L 7/10; A23L 7/196; A23L 11/00; A23L 25/00; A23P 20/15; A61K 9/00

(52) U.S. Cl. CPC A61K 38/39; A23F 5/02; A23F 5/14; A23L 7/101; A23L 7/196; A23L 11/01; A23L 25/20; A23P 20/15; A61K 9/0056

(57) ABSTRACT

The present disclosure is directed to infused nut, seed, legume, grain or coffee husk compositions and methods of making the same. For example, the present disclosure is directed to methods of fortifying nuts, seeds, legumes, grains and/or coffee husks. In some embodiments, the methods include drying a nut, seed, legume, grain or coffee husk, and infusing the nut, seed, legume, grain or coffee husk with a medium comprising water and one or more supplements. In some embodiments, the one or more supplements include a collagen, collagen derivative, collagen booster, or collagen alternative.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57, and Rules 4.18 and 20.6, including PCT Application No. PCT/US2024/053264 filed Oct. 28, 2024, and U.S. Provisional Application No. 63/594,125 filed Oct. 30, 2023, each of which is hereby incorporated by reference in its entirety.

### BACKGROUND

### Field

The present disclosure relates to methods of producing nut, seed, legume, grain and coffee husk food products improved by infusion of one or more supplements. More specifically, the present disclosure relates to a method of producing an improved and healthier nut, seed, legume and/or coffee husk food product infused with one or more supplements, wherein at least one supplement is a vitamin, nutrient, mineral, botanical, extract, hormone, herb, nutraceutical, lipid, carbohydrate, amino acid, protein, acid, salt, fungus, prebiotic, probiotic, microbial culture and/or other substance and compound.

### Description of the Related Art

The human body needs many kinds of nutrients, such as proteins, lipids, carbohydrates, vitamins, minerals, and water, to live and stay healthy but most industrial and commercial products, especially in North America are nutrient deficient and do not provide the necessary nutrients to support a healthy life. Individuals must buy several different kinds of foods, calculate each, and put them together in a specific way. This highly nutritious food can be consumed independently, it can also be used as a medium to deliver herbal remedies and nutritional supplements, as well as an additive or complement of other foodstuffs to enhance the nutritional value of these products.

Nuts and seeds are a type of food that has been consumed for hundreds of years and can be considered one of the oldest foods consumed by hunters and gatherers. Nowadays, there is a huge global demand for a variety of nuts, seeds, and legumes such as almonds, pistachios, peanuts, macadamia nuts, sunflower seeds, cashews, walnuts, hazelnuts, pecans, pine nuts, Brazil nuts, etc.

Nuts are known for having several health benefits such as improving artery health, reducing inflammation related to heart diseases, decreasing risks of blood clots, lowering the risk of high blood pressure, and lowering levels of low-density lipoprotein (LDL) cholesterol and triglycerides which is known to clog arteries. Furthermore, it is a known fact that oils contained in nuts, such as omega-3 fats have been shown to help prevent heart disease and stroke, may help control lupus, eczema, and rheumatoid arthritis, and may play protective roles in cancer and other conditions.

Legumes and seeds are known to be rich in nutrients, and to contribute to health applications such as heart health, weight management, blood sugar control, digestive health, bone health, cancer prevention, reduced risk of chronic diseases, better nutrient absorption, inflammation reduction, improved gut health, eye health, and a lowered risk of metabolic syndrome. Legumes and seeds are an excellent source of plant-based protein, making them a more environmentally sustainable protein choice.

Research is ongoing to explore the extraction of potentially beneficial compounds from coffee husks, such as antioxidants and dietary fibers. Coffee husks have also been used in applications such as compost, fertilizer, biomass fuel, animal feed, gardening, horticulture, mushroom cultivation, natural exfoliant, crafts, art, packaging material, biodegradable products, and construction material.

Other publications relate to the addition of flavor additives, fat substitutes, or an alternative fats (for example, a fat substitute or alternative fat lower in calories but which preserves the organoleptic properties of the nut) to nuts (including defatted nuts).

For example, WO 2013/085629 discloses a process to produce reduced fat, low-calorie nuts which involves applying pressure for several cycles at different pressures to a monolayer of nuts for a total duration of less than one minute, then applying a vacuum to the pressed nuts and oil, thus extracting the oil, the nut kernels are then reconstituted by contacting them with hot water at a temperature between 176° F. and 212° F. for less than one minute, and afterwards; annealing the reconstituted nut kernels by contacting them with a chilling cold medium, thereby producing hardened nut kernels.

U.S. Pat. No. 5,595,780 discloses a process in which partially defatted nuts, which have been defatted by pressing, are subjected to a vacuum of about 20 in Hg, then the nuts are contracted with an infusant in an amount of from 2 to 15% by weight of the nuts, the infusant being from 25 to 60% of an edible oil comprising additives selected from the group consisting of vitamins, flavoring agents and sweeteners; and from 40 to 75% of a second component which facilitates the formation of a desired nut color after roasting, and the infusant is absorbed into the nuts. The vacuum is then released. A pressure of 10 psi is then applied to the nuts and then released; the nuts are held for approximately one hour and then roasted. Thus, producing a flavor-improved and low-calorie nut product that has, allegedly, a higher yield.

### SUMMARY

Disclosed herein are methods to fortify nuts, seeds, legumes, grains or coffee husks with one or more supplements. In some embodiments, the methods include drying a nut, seed, legume, grain or coffee husk to an equilibrium relative humidity (ERH) of about 0.1% to 60% ERH; and infusing the nut, seed, legume, grain or coffee husk with a medium comprising water, and one or more supplements. In some embodiments, the nut, seed, legume, grain or coffee husk comprises a coffee bean. In some embodiments, the one or more supplements comprise a vitamin, mineral, botanical, extract, hormone, herb, nutraceutical, lipid, carbohydrate, amino acid, protein, acid, salt, fungi, prebiotic, probiotic, or microbial culture. In some embodiments, the one or more supplements comprise collagen, a collagen derivative, a collagen booster, a collagen alternative, or a combination thereof. In some embodiments, the collagen derivative comprises granulated hydrolyzed collagen peptides.

In some embodiments, the nut, seed, legume, grain or coffee husk is dried to an equilibrium relative humidity (ERH) of about 0.1% to 15% ERH. In some embodiments, infusing comprises mixing the medium and the nut, seed, legume, grain or coffee husk by recirculating the medium, pumping the medium, or agitating a mixture of the medium and the nut, seed, legume, grain or coffee husk. In some embodiments, infusing comprises allowing the nut, seed, legume, grain or coffee husk to absorb the medium for a period of 10 minutes to 24 hours. In some embodiments, the medium further comprises a surfactant, enzyme, or emulsifier. In some embodiments, drying comprises adding heat to or subtracting heat from the nut, seed, legume, grain or coffee husk. In some embodiments, drying comprises adding air or other gasses to or subtracting air or other gasses from the nut, seed, legume, grain or coffee husk. In some embodiments, the nut, seed, legume, grain or coffee husk is sterilized. In some embodiments, the nut, seed, legume, grain or coffee husk is left to infuse until a desired level of infusion or absorption is achieved. In some embodiments, the nut, seed, legume, grain or coffee husk is defatted. In some embodiments, the nut, seed, legume, grain or coffee husk is whole, powdered, ground, crushed, sliced, chopped, husked, de-husked, or is a part of a whole nut, whole seed, whole legume, grain or whole coffee husk.

In some embodiments, the collagen, collagen derivative, collagen booster, collagen alternative, or combination thereof is of bovine, marine, fish, porcine, or chicken origin. In some embodiments, the collagen, collagen derivative, collagen booster, collagen alternative, or combination thereof comprises gelatin, hydrolyzed collagen peptides, pure collagen, partially pure collagen, or a combination thereof.

In some embodiments, the method further includes washing the infused nut, seed, legume, grain or coffee husk. In some embodiments, the method further includes drying the infused nut, seed, legume, grain or coffee husk to an ERH of about 0.1% to about 20%.

In another aspect, disclosed herein are methods to infuse nuts, seeds, legumes, grains or coffee husks with one or more supplements. In some embodiments, the method includes drying the nuts, seeds, legumes, grains or coffee husks to an equilibrium relative humidity (ERH) of about 0.1% to 15% ERH; mixing the nuts, seeds, legumes, grains or coffee husks and a medium comprising water and one or more supplements; allowing the nuts, seeds, legumes, grains or coffee husks to absorb the medium, thereby infusing the nuts, seeds, legumes, grains or coffee husks; washing the infused nuts, seeds, legumes, grains or coffee husks with water; and drying the infused nuts, seeds, legumes, grains or coffee husks to 0.1-20% ERH. In some embodiments, the nuts, seeds, legumes, grains or coffee husks comprise coffee beans. In some embodiments, the coffee beans comprise whole green coffee beans, and wherein the whole green coffee beans are dried to an ERH of about 0.1% to about 10% before infusion.

According to a further aspect, disclosed herein are systems for infusing granular solids. In some embodiments, the system includes a tank comprising: an opening configured to receive granular solids and one or more liquids; an infusion chamber in fluidic communication with the opening; a liquid drain in fluidic communication with the infusion chamber; and an outlet in fluidic communication with the infusion chamber; an air source in fluidic communication with the infusion chamber; and an auger positioned interior to the infusion chamber.

In some embodiments, the system further comprises a lid positioned over the opening. In some embodiments, the air source comprises a fan. In some embodiments, the air source comprises an aeration pump. In some embodiments, the auger is in mechanical communication with a variable speed motor. In some embodiments, the system further includes a heat source in thermal communication with the infusion chamber. In some embodiments, the heat source comprises an infrared heater. In some embodiments, the system further comprises a water source in fluidic communication with the infusion chamber. In some embodiments, the water source comprises a spray head. In some embodiments, the air source comprises an aeration pump and a fan. In some embodiments, the fan is a variable speed fan.

In some embodiments, the granular solids comprise nuts, seeds, legumes, grains or coffee husks. In some embodiments, the seeds comprise coffee beans. In some embodiments, the one or more liquids comprise a medium comprising water, collagen, a collagen derivative, a collagen booster, a collagen alternative, one or more supplements, or a combination thereof.

According to a further aspect, disclosed herein are methods to fortify nuts, seeds, legumes, grains or coffee husks with one or more supplements. In some embodiments, the method includes disposing a nut, seed, legume, grain, or coffee husk in an infusion chamber; drying a nut, seed, legume, grain or coffee husk to an equilibrium relative humidity (ERH) of about 0.1% to 60% ERH; infusing the nut, seed, legume, grain, or coffee husk with a medium comprising water, and one or more supplements in the infusion chamber; and drying the infused nut, seed, legume, grain, or coffee husk in the infusion chamber.

In some embodiments, drying the nut, seed, legume, grain or coffee husk to the equilibrium relative humidity (ERH) of about 0.1% to 60% ERH comprises: mixing and agitating the nut, seed, legume, grain, or coffee husk via an auger rotating within the infusion chamber; supplying an air flow to the infusion chamber via a fan; and supplying heat to the infusion chamber via a heat source.

In some embodiments, infusing the nut, seed, legume, grain, or coffee husk with the medium comprising water and the one or more supplements comprises: preparing the medium comprising water and one or more supplements; disposing the medium in the infusion chamber; and mixing the medium with the nut, seed, legume, grain, or coffee husk disposed in the infusion chamber via an auger.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear. In addition to the features described herein, additional features and variations will be readily apparent from the following descriptions of the drawings and exemplary embodiments. It is to be understood that these drawings depict typical embodiments, and are not intended to be limiting in scope.

FIG. 1A is a flow diagram that schematically illustrates methods of fortifying nuts, seeds, legumes, grains or coffee husks.

FIG. 1B is a flow diagram that further schematically illustrates a process of infusing a nut, seed, legume, grain or coffee husk in a medium comprising water and one or more supplements, such as collagen, a collagen derivative, a collagen booster, a collagen alternative, or a combination thereof, taking place within the method of FIG. 1A.

FIG. 2 schematically illustrates embodiments of methods of fortifying nuts, seeds, legumes, grains or coffee husks.

FIG. 3A illustrates a schematic perspective view of an exemplary system.

FIG. 3B illustrates a schematic section view of the exemplary system of FIG. 3A.

FIG. 3C illustrates a top view of the exemplary system of FIG. 3A.

FIG. 4A illustrates a schematic perspective view of further exemplary system.

FIG. 4B illustrates a schematic section view of the exemplary system of FIG. 4A.

FIG. 4C illustrates a top view of the exemplary system of FIG. 4A.

FIG. 5A is a flow diagram that schematically illustrates methods of fortifying nuts, seeds, legumes, grains or coffee husks.

FIG. 5B is a flow diagram that further schematically illustrates a process of drying the nut, seed, legume, grain or coffee husk that takes place within the method of FIG. 5A.

FIG. 5C is a flow diagram that further schematically illustrates a process of infusing the nut, seed, legume, grain, or coffee husk with a medium comprising water and one or more supplements, that takes place within the method of FIG. 5A

### DETAILED DESCRIPTION

The foregoing and other aspects of the present disclosure will now be described in more detail with respect to the description and methodologies provided herein. This description is not intended to be a detailed catalogue of all the ways in which the embodiments of the present disclosure may be implemented, or of all the features that may be added to the present disclosure. For example, features illustrated with respect to one embodiment may be incorporated into other embodiments, and features illustrated with respect to a particular embodiment may be deleted from that embodiment. In addition, numerous variations and additions to the various embodiments suggested herein, which do not depart from the instant disclosure, will be apparent to those skilled in the art in light of the instant detailed description, figures and claims. Hence, the following specification is intended to illustrate some particular embodiments, and not to exhaustively specify all permutations, combinations and variations thereof.

All patents, patent applications, and other publications, including all sequences disclosed within these references, referred to herein are expressly incorporated herein by reference, to the same extent as if each individual publication, patent or patent application was specifically and individually indicated to be incorporated by reference. All documents cited are, in relevant part, incorporated herein by reference in their entireties for the purposes indicated by the context of their citation herein. However, the citation of any document is not to be construed as an admission that it is prior art with respect to the present disclosure.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

### Definitions

Although the following terms are believed to be well understood by one of skill in the art, the following definitions are set forth to facilitate understanding of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art.

As used herein, the terms “a” or “an” or “the” may refer to one or more than one. For example, “a” marker can mean one marker or a plurality of markers.

As used herein, the term “about,” when used in reference to a measurable value such as an amount of mass, dose, time, temperature, and the like, is meant to encompass variations of 20%, 10%, 5%, 1%, 0.5%, or even 0.1% of the specified amount.

As used herein, the term “and/or” refers to and encompasses any and all possible combinations of one or more of the associated listed items, as well as the lack of combinations when interpreted in the alternative (“or”).

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

As used herein, the term “consists essentially of” (and grammatical variants thereof), as applied to the compositions and methods of the present disclosure, means that the compositions/methods may contain additional components so long as the additional components do not materially alter the composition/method. The term “materially alter,” as applied to a composition/method, refers to an increase or decrease in the effectiveness of the composition/method of at least about 20% or more. For example, a component added to a composition of the present disclosure would “materially alter” the composition if it increases or decreases the composition's ability to inhibit tumor growth by at least 20%.

As used herein, “coffee husk” may refer to the protective outer layer that surrounds the coffee bean within the coffee cherry. When coffee cherries are processed to extract the coffee beans, the husks are removed as a byproduct. Coffee husks are lightweight and have a fibrous texture. A coffee husk of the coffee cherry or coffee fruit may be derived from a natural or dry processing method, and may be known as “coffee cascara,” coffee chaff, or coffee parchment by those skilled in the art.

As used herein, “collagen” may refer to the fibrous, abundant, and structurally intricate protein present in the extracellular matrix of various tissues in vertebrates, including type I, and type II collagen. Collagen may include pure collagen or partially pure collagen.

As used herein, “collagen derivative” may refer to a modified or altered form of collagen that has undergone chemical, enzymatic, or structural modifications from its original state. Examples of collagen derivatives include cross-linked collagen, collagen peptides (such as hydrolyzed collagen peptides), and collagen-based biomaterials, and gelatin.

As used herein, “collagen booster” may refer to a substance used to enhance the body's natural production of collagen or to protect existing collagen from degradation. Examples of collagen boosters may include vitamins (like vitamin C), antioxidants, minerals (like zinc), amino acids (like proline and lysine), or compounds that are believed to support collagen formation.

As used herein, “collagen alternative” may refer to a substance used in place of traditional collagen, for example due to ethical, environmental, or functional reasons. Examples of collagen alternatives include plant-based collagen alternatives (including extracts from algae, soy, bamboo, aloe vera, or fruits, including plant peptides) or synthetic collagen.

Without being bound by theory, “infusion” as understood in the present disclosure can generally be described as inserting or adding one or more substances (such as one or more supplements and/or cannabinoids, flavors and additives) to a food product, such as a nut, seed, legume, grain or coffee husk. “Infusion” is chemically independent in that its purpose is not to chemically alter the food product, but rather merely to result in the one or more substances being absorbed into and then residing inside the food product. In some embodiments, an infusion may typically take less than 12 hours of contact between the food product and the one or more substances to infuse the food product with the one or more substances. In some embodiments, infusion results in introducing or adding a certain modifying element or quality. In some embodiments, “fusion,” may refer to combining two or more things together in one; the act of combing things to form a new whole. In some embodiments, the methods and systems herein facilitate adding supplements (infusion/infused) to nuts, seeds, legumes, grains or coffee husks, and the advanced process capabilities can, in some embodiments, create a new whole (fusion/fused) product.

In contrast, and without being bound by theory, “fermentation” is a metabolic process that produces chemical changes in organic substances through the action of enzymes. In biochemistry, fermentation refers to the extraction of energy from carbohydrates in the absence of oxygen. “Fermentation” is chemically bound in that the food product is chemically altered, and one or more substances may become part of the food product. For example, a food product may be fermented alongside one or more substances, and as a result, the food product is chemically altered. Fermentation typically takes 12 to 120 hours or even longer depending on the product.

In colloquial use, fermentations are many times loosely called infusions, however this may be technically incorrect. Confusion in terminology may come from translation across multiple languages, or usage by less technical speaking people with most, almost all, fermentations coming from the farm level.

Advanced infusions have not, to date, been used in the field of nuts, seeds, legumes, grains or coffee husks, and specifically coffee beans, for several possible reasons. First, coffee farmers and supporting organizations may be opposed to any processes and/or substances that are not naturally produced or applicable in their natural coffee producing processes. Second, approximately 95% of coffee produced in 3rd world countries. This reality may create a disconnect between farmers, organizations and manufactures. Furthermore, available soluble materials (supplements) may be limited. Finally, the field may have not yet recognized a need to innovate as nuts, seeds (such as coffee beans), legumes and coffee husks have been highly healthy and flavorful on their own. However, with nutrition becoming more critical in view of the modern diet, infusion of nuts, seeds (such as coffee beans), legumes and coffee husks with one or more supplements has become necessary to provide additional health and wellness benefits.

As use herein the term “fortify” may refer to enriching a food substance (for example, such as a nut, seed, legume, grain or coffee husk) by adding ingredients (such as one or more supplements, such as vitamins or minerals) to improve the nutritional value. In some embodiments, fortifying includes infusion. In some other embodiments, fortifying includes fusion.

As used herein, a “supplement” includes but is not limited to a vitamin, non-vitamin, nutrient, mineral, non-mineral, botanical, extract, hormone, herb, nutraceutical, lipid, carbohydrate, amino acid, protein, acid, salt, fungus, prebiotic, probiotic, botanical compound, or microbial culture.

As used herein, “vitamin” includes but is not limited to vitamin A, vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folate), vitamin B12 (cobalamin), vitamin C, vitamin D2 (ergocalciferol), vitamin D3 (cholecalciferol), vitamin E (tocopherol), vitamin K1 (phylloquinone), vitamin K2 (menaquinone).

As used herein, “mineral” includes but is not limited to calcium, potassium, sodium, magnesium, phosphorus, iron, zinc, copper, iodine, selenium, manganese, fluoride, chromium, molybdenum, sulfur, chloride, cobalt, vanadium, nickel, and silicon.

As used herein, “nutrient” includes but is not limited to carbohydrates, proteins, fats, vitamins (such as vitamin C and vitamin D), minerals (such as calcium and iron), fiber, water, antioxidants (such as flavonoids and carotenoids), and essential fatty acids (such as omega-3 and omega-6).

As used herein, “botanical” refers to a dietary supplement made from plants or plant parts, such as herbs, flowers, leaves, seeds, roots, or stems. Botanical includes but is not limited to echinacea, ginger, ginseng, St. John's wort, turmeric, aloe vera, Ginkgo biloba, milk thistle, saw palmetto, valerian root, chamomile, lavender, ashwagandha, ginger, peppermint, fenugreek, garlic, dandelion, black cohosh, eucalyptus, licorice root, basil, evening primrose, Rhodiola rosea, cinnamon, cannabis, and elderberry.

As used herein, “botanical compound” refers to an active compound extracted from a plant. Botanical compounds include but are not limited to caffeine, curcumin (e.g., from turmeric), allicin (e.g., from garlic), flavonoids (e.g., from fruits and vegetables), silymarin (e.g., from milk thistle), resveratrol (e.g., from grapes), epigallocatechin gallate (EGCG, e.g., from green tea), ginsenosides (e.g., from ginseng), berberine (e.g., from goldenseal and other plants), quercetin (e.g., from apples and onions), anthocyanins (e.g., from berries), eugenol (e.g., from cloves), rosmarinic acid (e.g., from rosemary), glycyrrhizin (e.g., from licorice root), lutein (e.g., from marigold flowers), menthol (e.g., from peppermint), thymol (e.g., from thyme), capsaicin (e.g., from chili peppers), beta-carotene (e.g., from carrots), limonene (e.g., from citrus peels), tannins (e.g., from tea and wine), cannabidiol (CBD) and tetrahydrocannabinol (THC) (e.g., from cannabis). As used herein, “fungus” includes mushrooms and mycelium, including but not limited to Reishi (Ganoderma lucidum), Lion's Mane (Hericium erinaceus), Chaga (Inonotus obliquus), Cordyceps, and Turkey Tail (Trametes versicolor).

As used herein, “nut” may refer to a dry, indehiscent fruit that typically has a hard outer shell or pericarp that does not split open to release the seed inside when it matures, and may be formed from a single carpel of a flower. Examples of nuts include (but are not limited to) acorns, chestnuts, hazelnuts, beech nuts, macadamia nuts, hickory nuts, coco de mer, cola nut, or a combination of any of these.

As used herein, “seed” may refer to a mature ovule of a flowering plant (angiosperm) or gymnosperm that contains the embryonic plant along with a supply of stored food, enclosed within a protective seed coat. Examples of seeds include (but are not limited to) sunflower seed, pumpkin seed, watermelon seed, chia seed, sesame seed, flaxseed, poppy seed, mustard seed, quinoa seed, rice seed, wheat kernel, corn kernel, oat grain, barley grain, rye grain, soybean seed, pea seed, bean seed (such as kidney bean and black bean), lentil seed, almond nut (seed inside a drupe), walnut nut (seed inside a drupe), cashew nut (seed inside a cashew apple), pistachio nut (seed inside a drupe), pomegranate seed, strawberry seed (achene), raspberry seed (achene), blueberry seed (berry-like fruit), apple seed (in the core of the fruit), orange seed (in the flesh of the fruit), tomato seed (in the flesh of the fruit), mango seed (large seed in the center of the fruit), pine cone seed (conifer seed), maple samara (winged seed), dandelion seed (achene with a pappus for wind dispersal), coconut seed (inside the coconut fruit), cotton seed (inside the cotton boll), pepper seed (in the core of the pepper fruit), carrot seed (small and enclosed in the flower head), onion seed (inside the flower head), sunflower “kernel” (a type of achene), coffee bean (seed inside a coffee cherry), cocoa bean (seed inside a cocoa pod), buckwheat seed, millet seed, hemp seed, pecan nut (seed inside a drupe), Brazil nut (seed inside a capsule), hazelnut (filbert) nut (seed inside a husk), pine nut (seed inside a pine cone), macadamia nut (seed inside a hard shell), beech nut (seed inside a beech nut cupule), cumin seed, coriander seed, fennel seed, cardamom seed, black pepper seed (peppercorn), cucumber seed (in the core of the cucumber fruit), melon seed (inside melon fruit), avocado seed (large seed in the center of the fruit), fig seed (achene-like structure inside fig fruit), or a combination of any of these.

As used herein, “legume” may refer the fruit that typically consists of a pod containing seeds, from a plant belonging to the family Fabaceae (formerly known as Leguminosae). Examples of legumes include (but are not limited to): common bean (Phaseolus vulgaris), kidney bean (Phaseolus vulgaris), black bean (Phaseolus vulgaris), pinto bean (Phaseolus vulgaris), navy bean (Phaseolus vulgaris), lima bean (Phaseolus lunatus), chickpea (Cicer arietinum), lentil (Lens culinaris), green pea (Pisum sativum), snow pea (Pisum sativum var. saccharatum), snap pea (Pisum sativum var. macrocarpon), mung bean (Vigna radiata), adzuki bean (Vigna angularis), black-eyed pea (Vigna unguiculata), cowpea (Vigna unguiculata), soybean (Glycine max), broad bean (Vicia faba), fava bean (Vicia faba), red clover (Trifolium pratense), white clover (Trifolium repens), alfalfa (Medicago sativa), lupin (Lupinus spp.), winged bean (Psophocarpus tetragonolobus), pigeon pea (Cajanus cajan), lablab bean (Lablab purpureus), guar bean (Cyamopsis tetragonoloba), velvet bean (Mucuna pruriens), jack bean (Canavalia ensiformis), chickling vetch (Lathyrus sativus), French bean (Phaseolus vulgaris, also known as green bean or string bean), sword bean (Canavalia gladiata), moth bean (Vigna aconitifolia), yam bean (Pachyrhizus erosus), winged bean (Psophocarpus tetragonolobus), velvet bean (Mucuna pruriens), pigeon pea (Cajanus cajan), peanut (Arachis hypogaea), cluster bean (Cyamopsis tetragonoloba), cluster pea (Pisum sativum var. arvense), pinto peanut (Arachis pintoi), garden pea (Pisum sativum var. sativum), pea vetch (Lathyrus sativus), tepary bean (Phaseolus acutifolius), or a combination of any of these.

As used herein, “grain” may refer to a small, hard, one-seeded fruit of a cereal grass. Cereal grains may include the seeds of plants belonging to the grass family Poaceae (or Gramineae). Examples of grains include (but are not limited to): wheat, rice, oats, barley, corn, rye, quinoa, millet, sorghum, bulgur, buckwheat, teff, spelt, farro, amaranth, kamut, fonio, wild rice, freekeh, and emmer.

### Embodiments of Methods

In one aspect, described herein are methods of fortifying nuts, seeds, legumes, grains or coffee husks, including methods of infusing nuts, seeds, legumes, grains or coffee husks with one or more supplements such as collagen, a collagen derivative, a collagen booster, a collagen alternative, or a combination thereof. In some embodiments, the nut, seed, legume, grain or coffee husk is a coffee bean. In some embodiments, the coffee bean is a green coffee bean. In some embodiments, the coffee bean is a roasted coffee bean. The methods described herein may also be used with objects (such as food or beverage related and/or plant-based objects) other than nuts, seeds, legumes, grains or coffee husks which have a density below 1000 kg/m³.

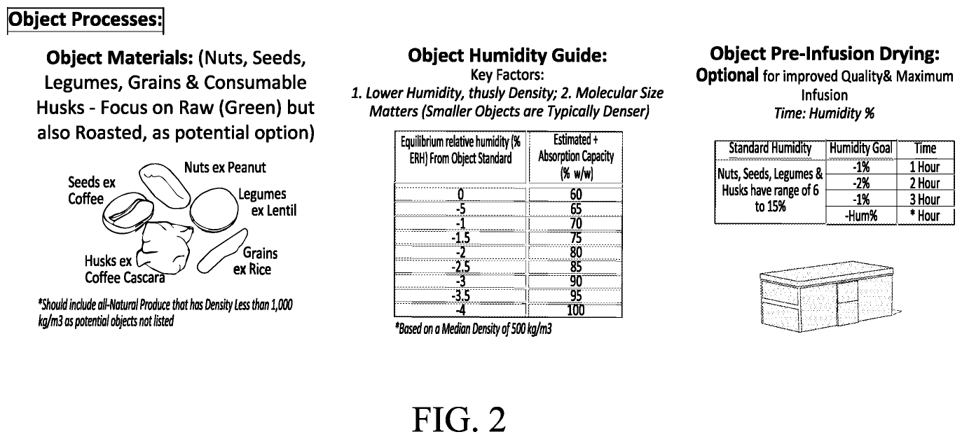

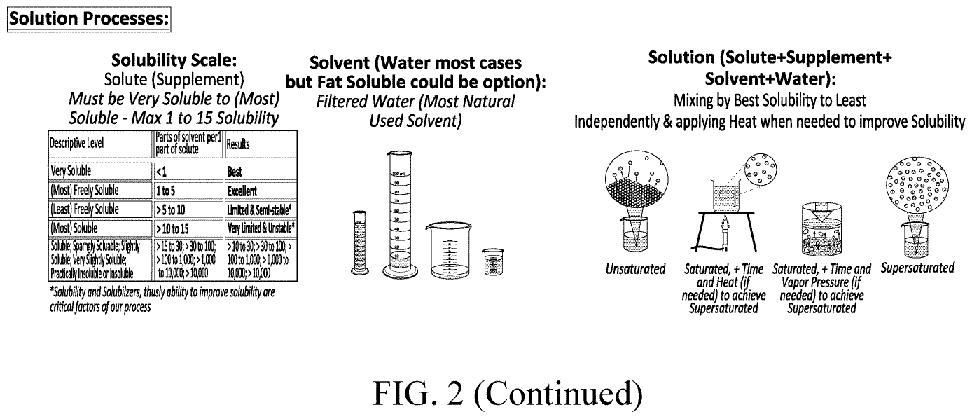

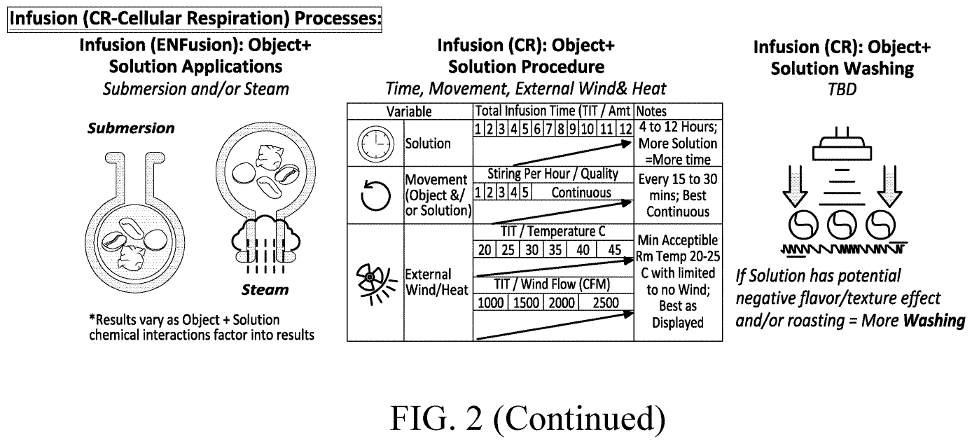

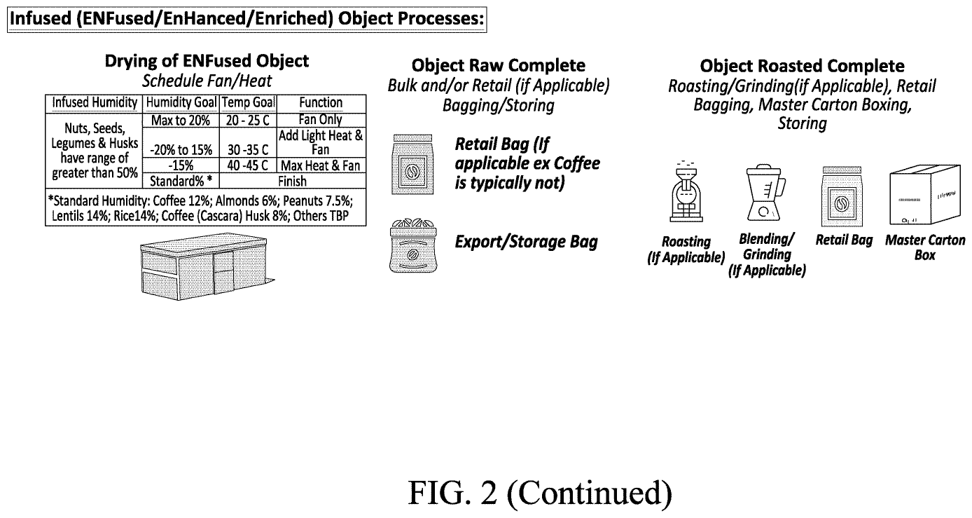

The methods of the present disclosure may have several advantages. For example, the infused nut, seed, legume, grain or coffee husk may provide for enhanced health of the eventual consumer. The methods provide for convenient delivery of one or more supplements. The methods provide for the creation of infused nut, seed, legume, grain or coffee husk that maintains more (if not all) natural flavor of the nut, seed, legume, grain or coffee husk, compared to methods of the prior art. Finally, in some embodiments, the methods provide for infusion via natural infusion process, and the nut, seed, legume, grain or coffee husk is not blended, maintaining the natural structure and appearance of the nut, seed, legume, grain or coffee husk. FIG. 2 schematically illustrates embodiments of methods of fortifying nuts, seeds, legumes, grains or coffee husks.

### Methods of Fortifying Nuts, Seeds, Legumes, Grains or Coffee Husks

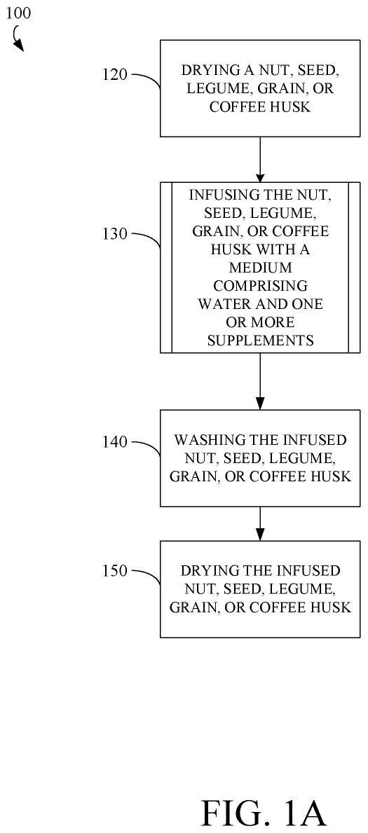

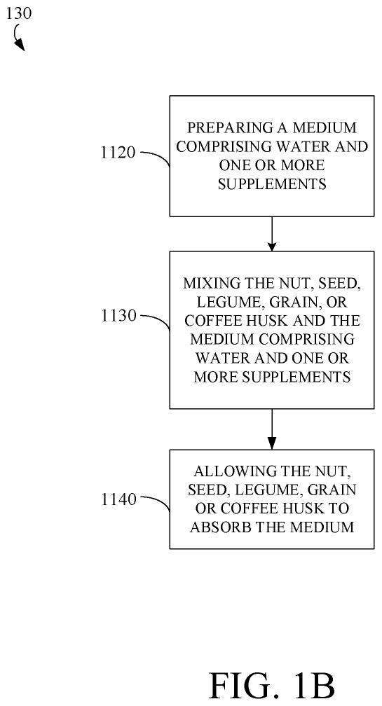

FIG. 1A is a block diagram that schematically illustrates an exemplary method 100 of fortifying nuts, seeds, legumes, grains or coffee husks.

### Drying a Nut, Seed, Legume, Grain or Coffee Husk

As shown in FIG. 1A, the method 100 for fortifying nuts, seeds, legumes, grains or coffee husks may start at block 120, wherein a nut, seed, legume, grain or coffee husk is dried. In some embodiments, drying the nut, seed, legume, grain or coffee husk thereby increases absorption capacity of the nut, seed, legume, grain or coffee husk.

In some embodiments, the nut, seed, legume, grain or coffee husk comprises a coffee bean. In some embodiments, the coffee bean is a green coffee bean. In some embodiments, the coffee bean is a decaffeinated coffee bean. In some embodiments, the coffee bean is a decaffeinated green coffee bean.

In some embodiments, the nut, seed, legume, grain or coffee husk is dried to an equilibrium relative humidity (ERH) of about 0.1% to 60% ERH. For example, the nut, seed, legume, grain or coffee husk may be dried to an equilibrium relative humidity (ERH) of about 0.1%, about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60% ERH, or any value therebetween, or a range constructed from any of the aforementioned values. In some embodiments, the nut, seed, legume, grain or coffee husk is dried to an equilibrium relative humidity of about 0.1% to 15% ERH. In some embodiments, the nut, seed, legume, grain or coffee husk is dried to an equilibrium relative humidity of about 5% to 15% ERH. In some embodiments, the nut, seed, legume, grain or coffee husk is dried to an equilibrium relative humidity of about 8% to 10% ERH. In some embodiments, seeds (including coffee beans) are dried to an ERH of about 8 to about 10%. In some embodiments, nuts are dried to an ERH of about 9 to about 10%.

In some embodiments, the nut, seed, legume, grain or coffee husk is dried according to methods known to those of skill in the art to achieve a desired relative humidity of the nut, seed, legume, grain or coffee husk. For example, methods of drying may include sun drying, air drying, oven drying, dehydrator, freeze drying, microwave drying, spray drying, vacuum drying, drum drying, belt drying, smoke drying, radiant energy drying, or a combination of any of these. For example, one of skill in the art may select the parameters of time and intensity of drying depending on the product to be made and the raw material used. For example, coffee seeds (coffee beans) may be dried from a starting ERH of about 12%. Nuts and other seeds may be dried from other starting ERHs.

In some embodiments, the nut, seed, legume, grain or coffee husk is dried for a period of 1 to 24 hours. In some embodiments, the nut, seed, legume, grain or coffee husk to is dried for a period of about 5 minutes, about 10 minutes, about 20 minutes, about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, or a value therebetween, or range constructed from any of the aforementioned values. In some embodiments, the nut, seed, legume, grain or coffee husk is dried for a period of about 4 to about 6 hours.

In some embodiments, drying includes adding heat to the nut, seed, legume, grain or coffee husk. For example, the nut, seed, legume, grain or coffee husk may be heated according to methods known to those of skill in the art. In some embodiments, drying includes subtracting heat from the nut, seed, legume, grain or coffee husk. For example, the nut, seed, legume, grain or coffee husk may be freeze-dried according to methods known to those of skill in the art.

In some embodiments, drying includes adding air or other gasses to the nut, seed, legume, grain or coffee husk. For example, drying methods may include drying by evaporation using methods known to those of skill in the art. For example, air drying may include use of a fan or blower to circulate air around the nut, seed, legume, grain or coffee husk. In some embodiments, drying includes removing air or other gasses from the nut, seed, legume, grain or coffee husk. For example, drying methods may include drying in vacuo using methods known to those of skill in the art.

In some embodiments, the nut, seed, legume, grain or coffee husk is sterilized. For example, the nut, seed, legume, grain or coffee husk may be sterilized before, during, or after the drying process. Sterilization may be by any method known to those of skill in the art. For example, in some embodiments, the nut, seed, legume, grain or coffee husk is sterilized with UV light or x-rays. Other methods of sterilization include heat sterilization (such as boiling, pasteurization, canning, retort processing, and/or hot water immersion), steam sterilization (including autoclaving), dry heat sterilization (including baking), radiation sterilization (including ionizing radiation, and/or ultraviolet (UV) radiation), chemical sterilization, filtration, high pressure processing, pulsed electric field, osmotic dehydration, aseptic processing, or a combination of any of these.

In some embodiments, the nut, seed, legume, grain or coffee husk is defatted. For example, the nut, seed, legume, grain or coffee husk may be defatted before, during, or after the drying process. For example, the nut, seed, legume, grain or coffee husk may be partially or entirely defatted by methods known to those of skill in the art. For example, defatting may include blanching, roasting, pressing, solvent extraction, air classification, water classification, enzyme treatment, supercritical fluid extraction, chemical treatment, grinding, or a combination of any of these.

The nut, seed, legume, grain or coffee husk may take a variety of forms. For example, in some embodiments, the nut, seed, legume, grain or coffee husk is whole, powdered, ground, crushed, sliced, chopped, husked, de-husked, or a combination of any of these. In some embodiments, the nut, seed, legume, grain or coffee husk a part of a whole nut, whole seed, whole legume, grain or whole coffee husk (respectively). Processing of a whole nut, whole seed, whole legume, grain or whole coffee husk (for example, to make it powdered, ground, crushed, sliced, chopped, de-husked, or otherwise part of a whole nut, whole seed, whole legume, grain or whole coffee husk) may take place before, during, or after the drying process, according to methods known to those of skill in the art.

### Infusing the Nut, Seed, Legume, Grain or Coffee Husk

As shown in FIG. 1A, the method 100 may proceed to process block 130, wherein the nut, seed, legume, grain or coffee husk is infused with a medium comprising water, and one or more supplements. FIG. 1B is a block diagram that illustrates the details on the methods taking place within the process block 130, wherein the nut, seed, legume, grain or coffee husk is infused with a medium comprising water, and one or more supplements. In some embodiments the one or more supplements include a collagen, collagen derivative, collagen booster, or collagen alternative.

### Washing the Infused Nut, Seed, Legume, Grain or Coffee Husk

As shown in FIG. 1A, the method 100 may proceed to block 140, wherein the infused nut, seed, legume, grain or coffee husk is washed.

For example, washing may be performed to remove residual media dried on the outside of the nut, seed, legume, grain or coffee husk. For example, washing may be performed in a resource-efficient and/or time efficient manner according to methods known to those of skill in the art. For example, in some embodiments the infused nut, seed, legume, grain or coffee husk is washed with water, such as purified water.

The infused nut, seed, legume, grain or coffee husk may be washed using methods known to those of skill in the art. For example, the infused nut, seed, legume, grain or electrostatic washing, chemical washing, enzymatic washing, ozone washing, ionized water washing, high-pressure washing, vibration washing, or a combination of any of these.

### Drying the Infused Nut, Seed, Legume, Grain or Coffee Husk

As shown in FIG. 1A, the method 100 may proceed to block 150, wherein the infused nut, seed, legume, grain or coffee husk is dried.

The infused nut, seed, legume, grain or coffee husk may be dried using methods known to those of skill in the art. For example, in some embodiments the infused nut, seed, legume, grain or coffee husk is dried using any of the methods described with respect to block 120, including a combination of these methods. In some embodiments, drying is staged to not overly heat and reduce quality of the final product. For example, heat may be increased as humidity of the drying nut, seed, legume, grain or coffee husk lowers.

In some embodiments, the infused nut, seed, legume, grain or coffee husk is dried according to methods known to those of skill in the art to achieve a desired relative humidity of the nut, seed, legume, grain or coffee husk. For example, one of skill in the art may select the parameters of time and intensity of drying depending on the product to be made and the raw material used. For example, the final desired relative humidity may depend on the raw material used (for example nuts or seeds, coffee, cascara coffee, almonds, peanuts, etc.). For example, one of skill in the art may select methods of drying the infused nut, seed, legume, grain or coffee husk in order to obtain a product suitable for human consumption and/or organoleptic preservation.

In some embodiments, the infused nut, seed, legume, grain or coffee husk is dried to an equilibrium relative humidity (ERH) of about 0.1% to 60% ERH. For example, the infused nut, seed, legume, grain or coffee husk may be dried to an equilibrium relative humidity (ERH) of about 0.1%, about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60% ERH, or any value therebetween, or a range constructed from any of the aforementioned values. In some embodiments, the infused nut, seed, legume, grain or coffee husk is dried to an equilibrium relative humidity (ERH) of about 0.1% to about 20%. In some embodiments, the infused nut, seed, legume, grain or coffee husk is dried to an equilibrium relative humidity (ERH) of about 5% to about 20%. In some embodiments, the infused nut, seed, legume, grain or coffee husk is dried to an equilibrium relative humidity (ERH) of about 8% to about 12%. In some embodiments, an infused coffee bean is dried to about 12% ERH. In some embodiments, an infused nut (such as an almond) is dried to about 6% ERH. In some embodiments, an infused legume (such as a peanut) is dried to about 7.5% ERH. In some embodiments, an infused coffee husk is dried to about 8% ERH.

In some embodiments, the infused nut, seed, legume, grain or coffee husk to is allowed to dry for a period of about 5 minutes, about 10 minutes, about 20 minutes, about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours, about 51 hours, about 52 hours, about 53 hours, about 54 hours, about 55 hours, about 56 hours, about 57 hours, about 58 hours, about 59 hours, about 60 hours, about 61 hours, about 62 hours, about 63 hours, about 64 hours, about 65 hours, about 66 hours, about 67 hours, about 68 hours, about 69 hours, about 70 hours, or a value therebetween, or range constructed from any of the aforementioned values. In some embodiments, the infused nut, seed, legume, grain or coffee husk to is allowed to dry for no more than 60 hours.

Methods of Infusing Nuts, Seeds, Legumes, Grains or Coffee Husks with One or More Supplements

FIG. 1B is a block diagram that illustrates the details on the methods taking place within the process block 130 described above, wherein the nut, seed, legume, grain or coffee husk is infused with a medium comprising water and one or more supplements. In some embodiments the one or more supplements include a collagen, collagen derivative, collagen booster, or collagen alternative.

Various factors may influence the infusion process and the parameters used. These factors include: humidity of the nut, seed, legume, grain or coffee husk; baseline (water only) absorption capacity of water in the nut, seed, legume, grain or coffee husk; solubility of the one or more supplements (such as very soluble or freely soluble); heat; air flow; stirring; quantity of ingredients; and time. One of skill in the art will be able to determine the necessary parameters based on these factors.

### Preparing a Medium

As shown in FIG. 1B, the method of the process block 130 may start from block 1120, wherein a medium comprising water and one or more supplements is prepared.

In some embodiments, the one or more supplements comprise collagen, a collagen derivative, a collagen booster, a collagen alternative, or a combination thereof. The collagen or collagen derivative may include any form of collagen or collagen derivative known to those of skill in the art. For example, in some embodiments, the collagen or collagen derivative includes gelatin, hydrolyzed collagen peptides (HCP), pure collagen, partially pure collagen, or a combination thereof. The collagen or collagen derivative may be of any origin known to those of skill in the art. For example, in some embodiments, the collagen or collagen derivative is of bovine, marine, fish, porcine, or chicken origin. In some embodiments, the collagen or collagen derivative is hydrolyzed collagen peptides (HCP). In some embodiments, the hydrolyzed collagen peptides (HCP) is granular HCP.

In some embodiments, inclusion of collagen, a collagen derivative, or a combination thereof may have one or more advantages. For example, these substances may have a high solubility rate of approximately 1:3. Collagen and collagen derivatives may also include 19 amino acids (including 8 of 9 essential amino acids). Collagen and collagen derivatives may also have a neutral flavor. When used to infuse a coffee bean, collagen and collagen derivatives may also maintain with neutral flavor and increase pH inside the coffee bean, making coffee smoother.

The one or more supplements may include any supplement known to those of skill in the art. For example, in some embodiments, one or more supplements may include a vitamin, mineral, botanical, extract, hormone, herb, nutraceutical, lipid, carbohydrate, amino acid, protein, acid, salt, fungi, prebiotic, probiotic, microbial culture, or a combination of any of these. In some embodiments, the one or more supplements include biotin. In some embodiments, the one or more supplements include an amino acid.

In some embodiments, the one or more supplements comprise collagen, a vitamin, a mineral, a botanical, an herb, a botanical compound, an amino acid, an endocannabinoid, guarana, or a mushroom. In some embodiments, the mineral comprises magnesium, zinc, or iron. In some embodiments, the mushroom comprises cordyceps. In some embodiments, the botanical compound comprises caffeine or curcumin. In some embodiments, the amino acid comprises tryptophan, glutamine, L-arginine, In some embodiments, the one or more supplements comprise L-Citrulline, L-carnitine, L-Tyrosine, or N-Acetyl L-Tyrosine (NALT). In some embodiments, the supplement comprises paraxanthine.

In some embodiments, the medium comprises water. In some embodiments, the water is purified water. The amount of water used may be determined by the person of skill in the art. For example, the amount of water may be determined based upon one or more of: needed liquidity, the absorption capacity of the dried nut, seed, legume, grain or coffee husk, the required level to create an initial equal infusion, the mixing time and/or method, and the absorption time. The amount of water may also be determined based on the solubility of the one or more supplements. For example, supplements which are very soluble may be added at a 1:1 ratio with water, and supplements which are freely soluble may be added at a 1:5 ratio with water. Levels of solubility of the one or more supplements may be described as in the table below. In some embodiments, the one or more supplements are very soluble according to the table below. In some embodiments, the one or more supplements are freely soluble according to the table below. In some embodiments, the one or more supplements are soluble in a range from 1:1-1:5 (parts solute:parts solvent). In some embodiments, the solvent is water. In some embodiments, heat may be utilized to increase solubility. For example, the infusion medium may be heated. In some embodiments, the solute is water-soluble. In some embodiments, the solute is fat-soluble.

TABLE 1

| | Parts of solvent per 1 |
|---|---|
| Descriptive Level | part of solute (material) |
| Very Soluble | Less than 1 |
| Freely Soluble | From 1 to 10 |
| Soluble | From 10 to 30 |
| Sparingly Soluble | From 30 to 100 |
| Slightly Soluble | From 100 to 1000 |
| Very Slightly Soluble | From 1000 to 10,000 |
| Practically Insoluble, or Insoluble | More than 10,000 |

In some embodiments, the amount of one or more supplements used may be determined based on the estimated amount to be delivered in the final product. For example, the amount may be related to the daily value (DV) of each supplement and the serving size of the final product. The amount may also be related to anticipated loss during extraction. For example, for coffee beans, it may be estimated that output after steeping/brewing is approximately 33%. For other products which are directly consumed, the output may be estimated to be 100%.

In some embodiments, the medium further comprises a surfactant, enzyme, or emulsifier. Any surfactant, enzyme, or emulsifier known to those of skill in the art may be used. Examples of surfactants and/or emulsifiers include lecithin, mono- and diglycerides, polysorbates, soy protein isolate, gum arabic, xanthan gum, carrageenan, agar, pectin, sorbitan esters (span), DATEM (diacetyl tartaric acid esters of monoglycerides), cellulose gum, sodium stearoyl lactylate (SSL), propylene glycol esters of fatty acids (PGME), polyglycerol esters (PGE), and glycerol monostearate. Examples of enzymes include protease, amylase, lipase, cellulase, lactase, pectinase, bromelain, papain, transglutaminase, phytase, alpha-amylase, catalase, alpha-galactosidase, dipeptidyl peptidase-4 (DPP-4), xylanase, invertase, hemicellulase, or a combination of any of these.

In some embodiments, preparing the medium comprises heating, boiling, coiling, agitating, stirring, mixing the solvent and solutes. For example, in some embodiments, the one or more supplements are boiled with water. In some embodiments, additional water is added after boiling. In some embodiments, the medium is agitated to prevent recrystallization of the one or more supplements in solution.

While the block 1120 has been described with respect to a medium comprising water, other solvents known to those of skill in the art may alternatively or additionally be used in some situations, for example, for supplements that are not water soluble.

### Mixing a Nut, Seed, Legume, Grain or Coffee Husk and the Medium

As shown in FIG. 1B, the method of the process block 130 may proceed to block 1130, wherein the nut, seed, legume, grain or coffee husk and the medium comprising water and one or more supplements are mixed.

One of skill in the art may select the parameters of time and intensity of mixing depending on the product to be made and the raw material used. For example, methods of mixing may include stirring, agitation, blending, mixing tanks or vessels, kneading, whisking, homogenization, emulsification, milling, grinding, centrifugation, tumbling, drum mixing, high-shear mixing, fold mixing, planetary mixing, ribbon mixing, paddle mixing, spiral mixing, fluidization, vortex mixing, jet mixing, static mixing, rotary drum mixing, or a combination of any of these.

In some embodiments, mixing includes movement of the nut, seed, legume, grain or coffee husk relative to the medium during the infusion process. In some embodiments, mixing includes continuous movement over a period of 1 minute to 3 hours. In some embodiments, mixing is over a period of about 1 minute, about 5 minutes, about 10 minutes, about 20 minutes, about 30 minutes, about 40 minutes, about 50 minutes, about 60 minutes, about 70 minutes, about 80 minutes, about 90 minutes, about 100 minutes, about 110 minutes, about 120 minutes, about 130 minutes, about 140 minutes, about 150 minutes, about 160 minutes, about 170 minutes, about 180 minutes, or more, or a value therebetween, or a range constructed from any of the aforementioned values. In some embodiments, mixing is over a period of about 10 minutes to about 120 minutes. In some embodiments, mixing is over a period of about 30 minutes to about 60 minutes.

In some embodiments, mixing is periodic. For example, mixing may be every about 1 minute, about 5 minutes, about 10 minutes, about 20 minutes, about 30 minutes, about 40 minutes, about 50 minutes, about 60 minutes, or more, or a value therebetween, or a range constructed from any of the aforementioned values. In some embodiments, the nut, seed, legume, grain or coffee husk and medium are mixed every 15 minutes. In some embodiments, the nut, seed, legume, grain or coffee husk and medium are mixed every 30 minutes.

In some embodiments, mixing comprises recirculating the medium around the nut, seed, legume, grain or coffee husk. This may be accomplished by methods known to those of skill in the art. For example, the medium may be recirculated from the bottom to the top of a container, for example by a shower which collects medium at the bottom of a container and redistributes the medium on top of the nut, seed, legume, grain or coffee husk.

In some embodiments, mixing comprises agitating the nut, seed, legume, grain or coffee husk within the medium. This may be accomplished by methods known to those of skill in the art. For example, a stirrer, an auger, or an angled rotating tank similar to a concrete mixer may be used to agitate the mixture of the medium and the nut, seed, legume, grain or coffee husk.

In some embodiments, mixing comprises pumping the medium through the nuts, seeds, legumes, grains or coffee husks. This may be accomplished by methods known to those of skill in the art. For example, the medium and the nut, seed, legume, grain or coffee husk may be placed in a container which includes a pump to pump the medium through the nuts, seeds, legumes, grains or coffee husks in the container.

### Allowing the Nut, Seed, Legume, Grain or Coffee Husk to Absorb the Medium

As shown in FIG. 1B, the method of the process block 130 may proceed to block 1140, wherein the nut, seed, legume, grain or coffee husk to is allowed to absorb the medium.

In some embodiments, the nut, seed, legume, grain or coffee husk is allowed to absorb the medium for a period of 10 minutes to 24 hours. In some embodiments, the nut, seed, legume, grain or coffee husk to is allowed to absorb the medium for a period of about 5 minutes, about 10 minutes, about 20 minutes, about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, or a value therebetween, or range constructed from any of the aforementioned values. In some embodiments the nut, seed, legume, grain or coffee husk to is allowed to absorb the medium for more than 24 hours. In some embodiments, the period comprises about 2 to 12 hours. In some embodiments the nut, seed, legume, grain or coffee husk to is allowed to absorb the medium for 4 to 10 hours. In some embodiments the nut, seed, legume, grain or coffee husk to is allowed to absorb the medium for 4 to 12 hours. In some embodiments, the nut, seed, legume, grain or coffee husk is allowed to absorb the medium for no more than 24 hours.

One of skill in the art may select the parameters of time and conditions of absorption depending on the product to be made and the raw material used. For example, the nut, seed, legume, grain or coffee husk may be left to infuse until a desired level of infusion is achieved. For example, the pre-dried nut, seed, legume, grain or coffee husk may have an absorption capacity of 60-100% for water. For example, the time of absorption may depend on the amount of the one or more supplements used in the medium.

In some embodiments, the absorption capacity of the nut, seed, legume, grain or coffee husk may be relative to the humidity of the nut, seed, legume, grain or coffee husk after pre-drying. For example, for coffee beans, an exemplary relationship between humidity of coffee beans and absorption capacity is shown in the table below. By an absorption capacity of 100%, it is meant that 250 g of coffee beans would be capable of absorbing 250 g of pure water.

TABLE 2

| Equilibrium relative | Absorption |
|---|---|
| humidity (% ERH) | capacity (% w/w) |
| 12 | 60 |
| 11.5 | 65 |
| 11 | 70 |
| 10.5 | 75 |
| 10 | 80 |
| 9.5 | 85 |
| 9 | 90 |
| 8.5 | 95 |
| 8 | 100 |

For example, in some embodiments, if the medium includes 10-20% collagen or collagen derivative by weight, the nut, seed, legume, grain or coffee husk is allowed to absorb the medium for a period of about 4 to about 6 hours. For example, in some embodiments, if the medium includes 20-35% collagen or collagen derivative by weight, the nut, seed, legume, grain or coffee husk is allowed to absorb the medium for a period of about 8 to about 10 hours.

In some embodiments, the nut, seed, legume, grain or coffee husk is allowed to absorb the medium at temperature range of 20° C. up to 45° C., for example 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., or a range constructed from any of the aforementioned values. In some embodiments, the nut, seed, legume, grain or coffee husk is allowed to absorb the medium under a wind flow. For example, wind flow may be from 1000 to 2500 CFM, for example 1000 CFM, 1100 CFM, 1200 CFM, 1300 CFM, 1400 CFM, 1500 CFM, 1600 CFM, 1700 CFM, 1800 CFM, 1900 CFM, 2000 CFM, 2100 CFM, 2200 CFM, 2300 CFM, 2400 CFM, 2500 CFM, or a range constructed from any of the aforementioned values.

In some embodiments, allowing the nut, seed, legume, grain or coffee husk thereby allows the nut, seed, legume, grain or coffee husk to be infused with the one or more supplements.

In some embodiments, allowing the nut, seed, legume, grain or coffee husk to absorb the medium comprises agitating the mixture of the medium and the nut, seed, legume, grain or coffee husk. This may be accomplished using agitation methods further described herein, for example, using the systems described herein, or using methods known those of skill in the art. In some embodiments, allowing the nut, seed, legume, grain or coffee husk to absorb the medium comprises increasing airflow around the mixture of the medium and the nut, seed, legume, grain or coffee husk. This may be accomplished using agitation methods further described herein, for example, using the systems described herein, or using methods known those of skill in the art.

### Embodiments of Compositions

In one aspect, described herein are compositions. In some embodiments, the composition includes an infused nut, seeds, legume, grain or coffee husk produced by the methods described herein. In some embodiments, the nut, seed, legume, grain or coffee husk is a coffee bean.

For example, one aspect of the present disclosure is directed to an improved and novel infused nut or seed. Another aspect of the present disclosure is directed to an improved and novel infused coffee husk.

Compositions according to the present disclosure may be infused with at least one supplement, such as a health or fitness supplement, including but limited to, a vitamin, non-vitamin, mineral, non-mineral, botanical, extract, hormone, herb, nutraceutical, lipid, carbohydrate, amino acid, protein, acid, salt, fungi, prebiotic, probiotic, microbial culture and/or other substance and compound.

The compositions disclosed herein may improve the quality and/or experience of the life of the consumer, and potentially overcomes wholly or partially one or more drawbacks of the known regular consumption of nut, seed, and/or coffee husk. According to an embodiment, the proposed nut, seed and/or coffee husk provides the user with additional health benefits beyond the natural form of the nut, seed, and/or coffee husk, respectively, but retains the flavor of the natural form of the natural form of the nut, seed, and/or coffee husk, respectively.

### Systems

Further disclosed herein are systems and devices for implementing the methods described herein. In one aspect, described herein are systems for infusing granular solids with one or more supplements. In some aspects, the granular solids include nuts, seeds, legumes, grains or coffee husks. In some aspects, the nuts, seeds, legumes, grains or coffee husks with are infused with one or more supplements such as collagen, a collagen derivative, a collagen booster, a collagen alternative, or a combination thereof. In some aspects, the one or more supplements are a liquid, are emulsified in a liquid, or are dissolved in a liquid. In some aspects, the granular solid (e.g., nut, seed, legume, grain or coffee husk) is a coffee bean. In some aspects, the coffee bean is a green coffee bean. In some aspects, the coffee bean is a roasted coffee bean. The methods described herein may also be used with objects (such as food or beverage related and/or plant-based objects) other than nuts, seeds, legumes, grains or coffee husks which have a density below 1000 kg/m³.

The systems and devices of the present disclosure may have several advantages. For example, the infused nut, seed, legume, grain or coffee husk may provide for enhanced health of the eventual consumer. The systems allow for efficient batch processing of the infusion method within a single container. The systems provide for the creation of infused nut, seed, legume, grain or coffee husk that maintains more (if not all) natural flavor of the nut, seed, legume, grain or coffee husk, compared to systems of the prior art. Finally, in some embodiments, the systems provide for infusion via natural infusion process, and the nut, seed, legume, grain or coffee husk is not blended, maintaining the natural structure and appearance of the nut, seed, legume, grain or coffee husk.

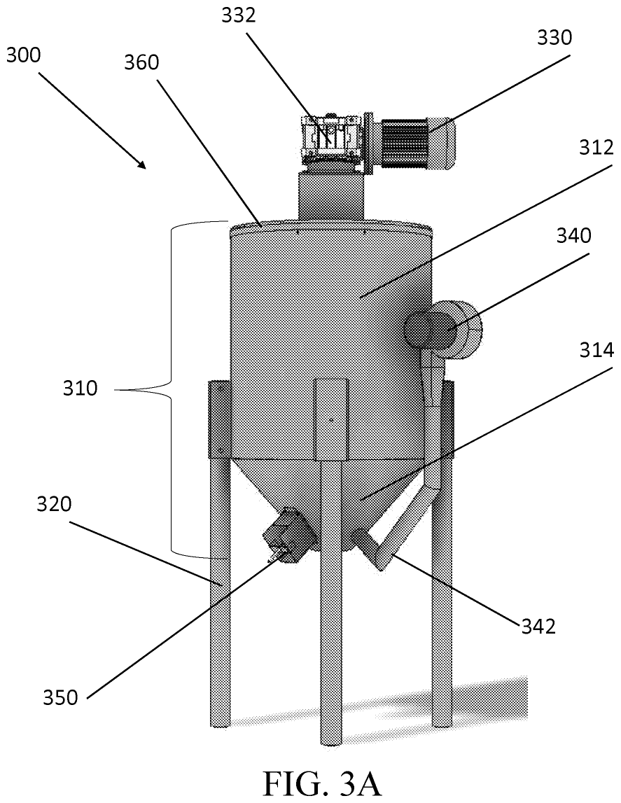

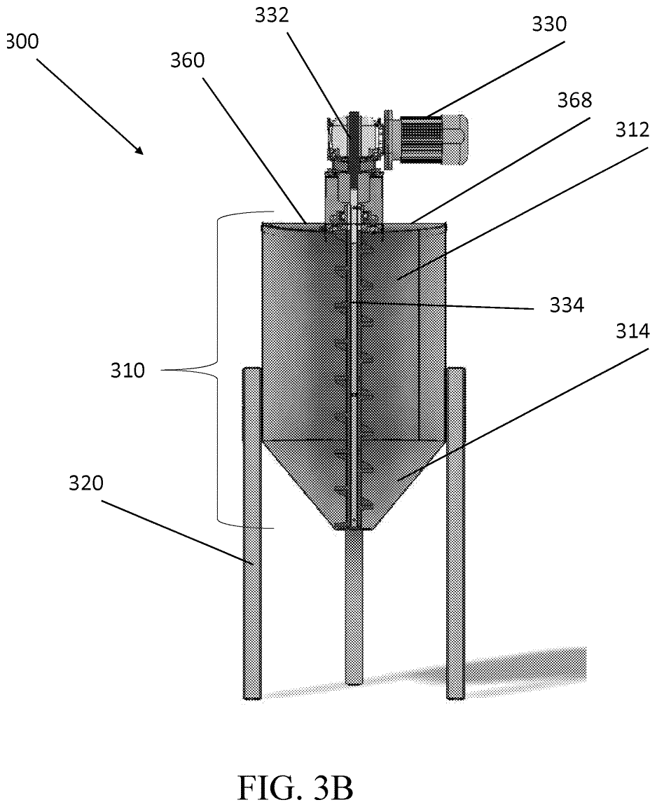

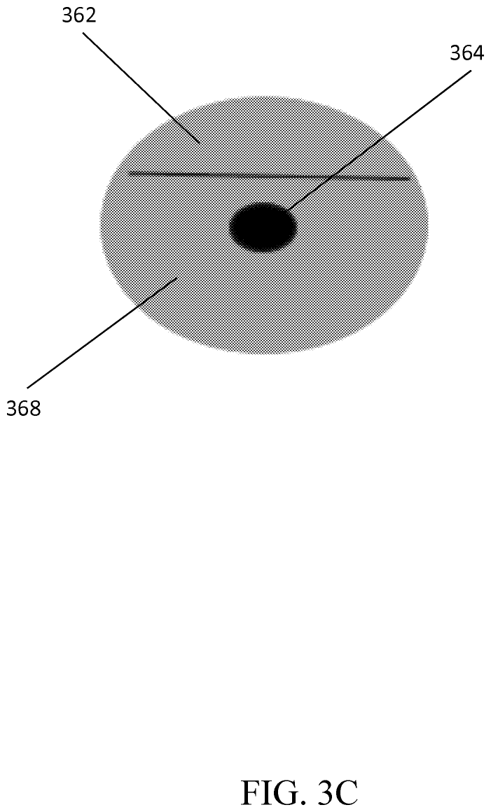

FIGS. 3A-3C illustrate an example of a system 300 for infusing granular solids with a liquid. In some embodiments, the system 300 described herein advantageously allows for reduced infusion time, a reduction or elimination of residue buildup or clumping during the infusion process, an increased ration of solution to granular solid during the infusion process, and increased oxygen availability during the infusion process. In some examples, the time required to infuse the nut, seed, legume, grain or coffee husk can be reduced by 100-400%.

FIG. 3A illustrates a schematic perspective view of the system 300. FIG. 3B illustrates a schematic section view of the system 300. FIG. 3C illustrates a top view of the system 300. As illustrated, the system 300 includes an infusion chamber 310. The infusion chamber 310 includes a cylindrical portion 312 and a conical cap 314 positioned at a bottom end of the cylindrical portion 312. In some examples the infusion chamber 310 can be rectangular, polygonal, pill shaped, trough shaped, or conical. In some examples, the infusion chamber 310 can have a total capacity of about 20 Kg, 30 Kg, 40 Kg, 50 Kg, 60 Kg, 70 Kg, 80 Kg, 90 Kg, 100 Kg, 200 kg, 300 kg, 400 kg, 500 kg, 600 kg, 700 kg, 800 kg, 900 kg, 1000 kg, 1500 kg, 2000 kg, 2500 kg, 3000 kg, 3500 kg, 4000 kg, 5000 kg, or more, or a range constructed from any of the aforementioned values. In some examples, the infusion chamber 310 can have a granular solid capacity of 10 Kg, 15 Kg, 20 Kg, 25 Kg, 30 Kg, 35 Kg, 40, Kg, 45 Kg, 50 Kg, or more. In some examples, the infusion chamber can have a solution capacity of 10 Kg, 15 Kg, 20 Kg, 25 Kg, 30 Kg, 35 Kg, 40, Kg, 45 Kg, 50 Kg, 55 Kg, 60 Kg, or more. The infusion chamber 310 is elevated and supported by a plurality of legs 320 which attach to the cylindrical portion 312 so that the conical cap 314 is elevated off the ground. As illustrated, the system 300 includes a plurality of legs 320, which in the illustrated embodiment of FIG. 3A is three legs. In some examples, 3, 4, 5 or more legs support the infusion chamber 310. In some examples, the infusion chamber 310 is supported by a unitary base.

An opening 360 is positioned at the top of the infusion chamber 310 (e.g., the opposite end from the conical cap 314. The opening 360 can span the perimeter of the cylindrical portion 312. The opening 360 can be configured to receive both granular solids and one or more liquids. The granular solids and one or more liquids can pass through the opening 360 and into the infusion chamber 310. A lid 368 is positioned over the opening 360. As illustrated in FIG. 3C, the lid 368 can include a hatch 362 which can open and close. The hatch 362 can be opened to add granular solids and/or liquids to the infusion chamber 310. The hatch 362 can be closed to protect the infusion chamber 310 from contamination. The lid 368 also includes a port 364 which a drive shaft 332 of an auger 334 can pass through. The port 364 is positioned in a structural portion of the lid 368 opposite from the hatch 362.

The auger 334 (best illustrated in FIG. 3B) is positioned along the central axis of the infusion chamber 310 and extends from the opening 360 to the bottom end of the conical cap 314. The auger 334 includes a drive shaft 332 which extends upward out of the opening 360 through the port 364. The drive shaft 332 is mechanically attached to a motor 330. In some examples, the motor 330 is attached and/or supported by the lid 368 around the port 364. The motor 330 can drive the drive shaft 332 causing the auger 334 to rotate. The auger 334 is configured to mix the granular solids and/or one or more liquids held in the infusion chamber. In some examples, the motor 330 is a variable speed motor. In some examples, the motor 330 is a single speed motor. In some examples, the motor 330 includes a clutch to reduce the risk of motor and/or auger damage in the case of a jam. In some examples, the auger 334 had a constant cross section helical blade. In some examples, the auger 334 has a conical cross section helical blade. In some embodiments, the helical blade of the auger 334 has a constant curvature. In some embodiments, the helical blade of the auger 334 has a variable curvature. In some embodiments, the helical blade of the auger 334 has a variable slope. In some embodiments, the helical blade of the auger 334 has a constant slope.

A fan 340 is attached to the exterior of the infusion chamber 310. An air channel 342 fluidically connects the fan 340 to the infusion chamber 310 so that the fan 340 can force air into the infusion chamber 310. In some examples, the fan 340 has a variable speed motor. In some examples, the fan 340 has a single speed motor. In some examples, the fan 340 is driven by the same motor 330 as the auger 334.

An outlet 350 is positioned on the conical cap 314. The outlet 350 can be configured to selectively release liquids and/or solids out of the infusion chamber. In some examples, the outlet 350 includes a removable screen which liquids can pass through, but solid granules are inhibited from passing through. In some examples, the removable screen can be removed to allow solid granules to exit the infusion tank.

In some examples, the opening 360 is configured to receive granular solids and a solution. The infusion chamber 310 is in fluidic communication with the opening 360 and the granular solids and solution pass through the opening 360 and into the infusion chamber 310. The auger 334 is positioned interior to the infusion chamber 310 and is configured to mix and agitate the granular solids and solution within the infusion chamber 310. The fan 340 is in fluidic communication with the infusion chamber 310 so that the fan 340 can supply air to the infusion chamber 310. The outlet 350 is in fluidic communication with the infusion chamber 310 so that any excess solution can exit the infusion chamber 310. The outlet 350 can also release the infused granular solids from the infusion chamber 310.

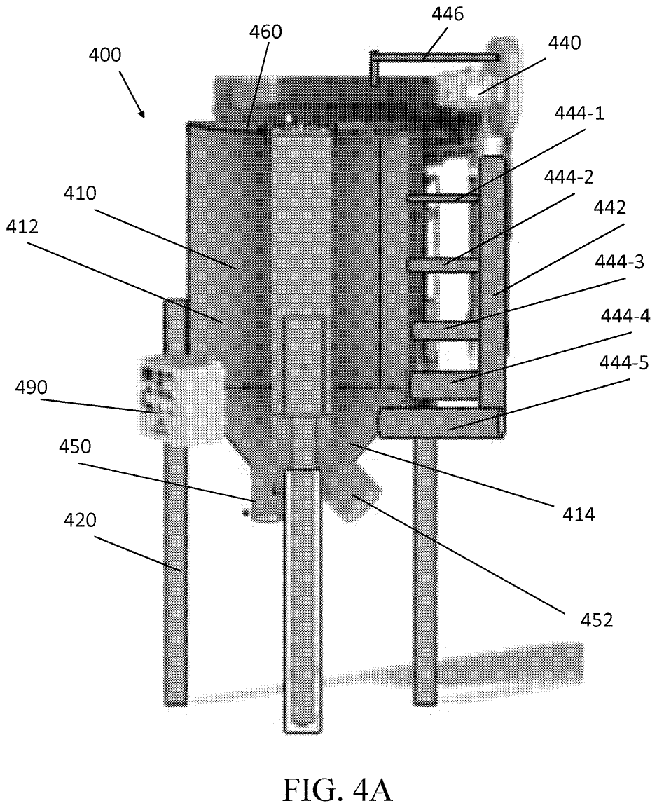

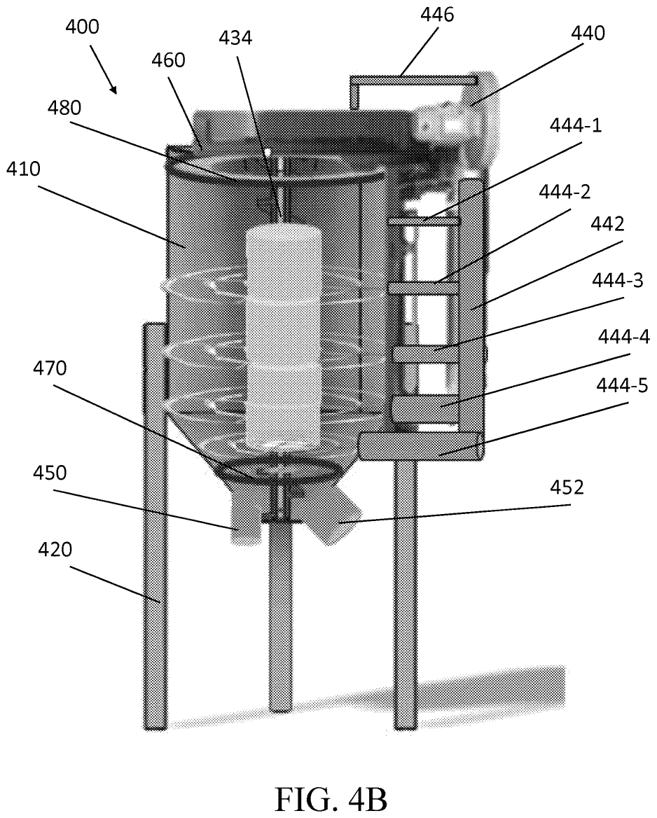

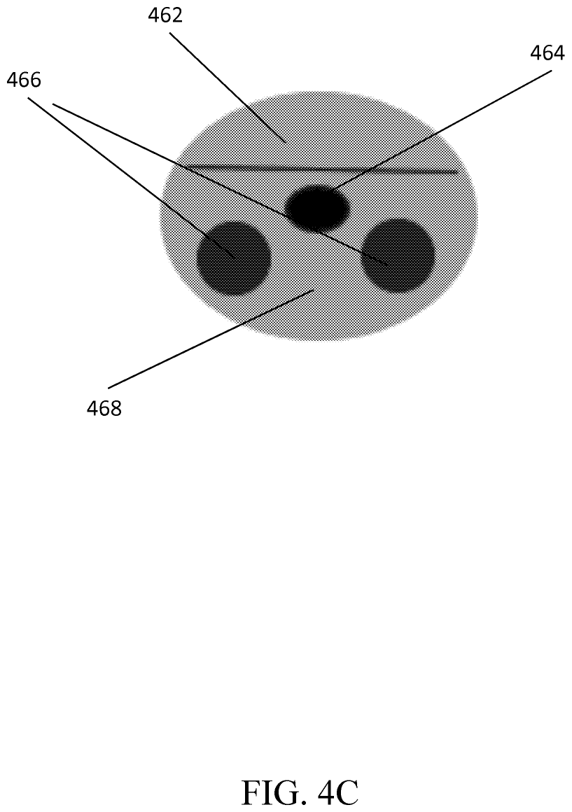

FIGS. 4A-4C illustrate an example of a system 400 for infusing granular solids with a liquid. In some embodiments, the system 400 described herein advantageously allows for reduced infusion time, a reduction or elimination of residue buildup or clumping during the infusion process, an increased ration of solution to granular solid during the infusion process, and increased oxygen availability during the infusion process. In some examples, the time required to infuse the nut, seed, legume, grain or coffee husk can be reduced by 100-400%.

FIG. 4A illustrates a schematic perspective view of the system 400. FIG. 4B illustrates a schematic section view of the system 400. FIG. 4C illustrates a top view of the system 400. As illustrated, the system 400 includes an infusion chamber 410. The infusion chamber 410 includes a cylindrical portion 412 and a conical cap 414 positioned at a bottom end of the cylindrical portion 412. The infusion chamber 410 can have a capacity of about 20 Kg, 30 Kg, 40 Kg, 50 Kg, 60 Kg, 70 Kg, 80 Kg, 90 Kg, or 100 Kg. In some examples, the infusion chamber 110 has a granular solid capacity of 10 Kg, 15 Kg, 20 Kg, 25 Kg, 30 Kg, 35 Kg, 40, Kg, 45 Kg, or 50 Kg. In some examples, the infusion chamber can have a solution capacity of 10 Kg, 15 Kg, 20 Kg, 25 Kg, 30 Kg, 35 Kg, 40, Kg, 45 Kg, 50 Kg, 55 Kg, or 60 Kg. The infusion chamber 410 is elevated and supported by a plurality of legs 420 which attach to the cylindrical portion 412 so that the conical cap 414 is elevated off of the ground. As illustrated, the system 400 includes a plurality of legs 420, which in the embodiment of FIG. 4A is three legs. In some examples, 3, 4, 5 or more legs support the infusion chamber 410. In some examples, the infusion chamber 410 is supported by a unitary base.

An opening 460 is positioned at the top of the infusion chamber 410 (e.g., the opposite end from the conical cap 414. The opening 460 can span the perimeter of the cylindrical portion 412. The opening 460 can be configured to receive both granular solids and one or more liquids. The granular solids and one or more liquids can pass through the opening 460 and into the infusion chamber 410. A lid 468 is positioned over the opening 460. As illustrated in FIG. 4C, the lid 468 can include a hatch 462 which can open and close. The hatch 462 can be opened to add granular solids and/or liquids to the infusion chamber 410. The hatch 462 can be closed to protect the infusion chamber 410 from contamination. The lid 468 also includes a port 464 which a drive shaft of an auger 434 can pass through. The port 464 is positioned in a structural portion of the lid 468 opposite from the hatch 462. A heat source 466 can be positioned on the structural portion of the lid 468 opposite from the hatch 462. The heat source 466 can be an infrared heat source. In some aspects, the heat source 466 can be temperature adjustable (e.g., dimmable).

The auger 434 (best illustrated in FIG. 4B) is positioned along the central axis of the infusion chamber 410 and extends from the opening 460 to the bottom end of the conical cap 414. The auger 434 includes a drive shaft (not pictured) which extends upward out of the opening 460 through the port 464. The drive shaft can mechanically attach to a motor (not pictured). In some examples, the motor is attached and/or supported by the lid 468 around the port 464. The motor can drive the drive shaft causing the auger 434 to rotate. The auger 434 is configured to mix the granular solids and one or more liquids held in the infusion chamber. In some examples, the auger 434 had a constant cross section helical blade. In some examples, the auger 434 has a conical cross section helical blade. In some embodiments, the helical blade of the auger 434 has a constant curvature. In some embodiments, the helical blade of the auger 434 has a variable curvature. In some embodiments, the helical blade of the auger 434 has a variable slope. In some embodiments, the helical blade of the auger 434 has a constant slope. In some embodiments, the motor is a variable speed motor, for example, a 10-speed motor.

A fan 440 is attached to the exterior of the infusion chamber 410. A plurality of air channels 444-1-444-5 fluidically connect the fan 440 to the infusion chamber 410 so that the fan 440 can force air into the infusion chamber 410. The uppermost air channel 444-1 has the narrowest flow path and the lowermost air channel 444-5 has the widest flow path, with each intermediary air channel being progressively wider down the length of the infusion chamber 410. The progressively increasing width of the air channels 444-1-444-5 can facilitate a greater air flow in the bottom of the infusion chamber 410 compared to the upper portion of the infusion chamber 410. A hot air channel 446 can feed into the fan 440 to provide heated air to each of the air channels 444-1-444-5. In some aspects, the fan 440 can be a variable speed fan motor. In some aspects, the same motor can drive both the fan 440 and the auger 434.

A sprayer 480 can be positioned interior to the infusion chamber 410. As illustrated, the sprayer 480 is positioned circumscribing the interior of the cylindrical portion 412 of the infusion chamber 410 adjacent to the opening 460. The sprayer 480 can be fluidically attached to a water line which can supply hot and/or cold to the infusion chamber 410. In some examples, the sprayer 480 can be attached to a fluid line which includes soaps and/or solvents. In some examples, the sprayer 480 can be selectively turned on and/or off. In some examples, the sprayer 480 can be selectively temperature controlled (e.g., have a “hot” mode and a “cold” mode or have temperature selection options).

An aeration pump 470 can be positioned interior to the infusion chamber 410. As illustrated, the aeration pump 470 is positioned circumscribing the interior of the conical cap 414. The aeration pump 470 can supply variable pressure air to the interior of the infusion chamber 410.

A liquid drain 450 is positioned on the conical cap 414. The liquid drain 450 can be configured to selectively drain liquids out of the infusion chamber 110. Granular solids can be inhibited from passing through the liquid drain 450. An outlet 452 is positioned on the conical cap 414. The outlet 452 can be configured to selectively drain solids out of the infusion chamber 410. In some examples, the liquid drain 450 can be fluidically attached to the sprayer 480 such that liquid from the infusion chamber 110 can be re-sprayed by the sprayer 480. In some examples, the sprayer 480 can redistribute the liquid from the infusion chamber 110 to the top of the granular solid in the infusion chamber 110.

In some examples, a user interface 490 can electrically communicate with a hardware processor to control the operation of the system 400. The hardware processor can execute program instructions to automatically control the operation of the system 400. For example, the hardware processor can set the auger rotation speed based on input from the user interface 490. The hardware processor can execute program instructions to set the aeration pump pressure based on input from the user interface 490. The hardware processor can execute program instructions to set the fan speed based on input from the user interface 490. The hardware processor can execute program instructions to turn off, turn on, and set the temperature of the wash/clean sprayer based on input from the user interface 490. The hardware processor can execute program instructions to open and/or close the liquid drain 450 based on input from the user interface 490. The hardware processor can execute program instructions to open and/or close the outlet 452 based on input from the user interface 490. In some examples, the hardware processor can have pre-programmed operational structures for performing any of the methods described herein. In some examples, the properties of the granular solid and the solutions placed in the infusion chamber 410 can be input into the user interface 490 and the hardware processor can implement a method described herein to infuse the granular solid

In some examples, the opening 460 is configured to receive granular solids and a solution. The infusion chamber 410 is in fluidic communication with the opening 460 and the granular solids and solution pass through the opening 460 and into the infusion chamber 410. The auger 434 is positioned interior to the infusion chamber 410 and is configured to mix and agitate the granular solids and solution within the infusion chamber 410. The fan 440 is in fluidic communication with the infusion chamber 410 so that the fan 440 can supply air to the infusion chamber 410. The liquid drain 450 is in fluidic communication with the infusion chamber 410 so that any excess solution can exit the infusion chamber 410. The outlet 452 is in fluidic communication with the infusion chamber 410 so that the outlet 452 can release the infused granular solids from the infusion chamber 410.

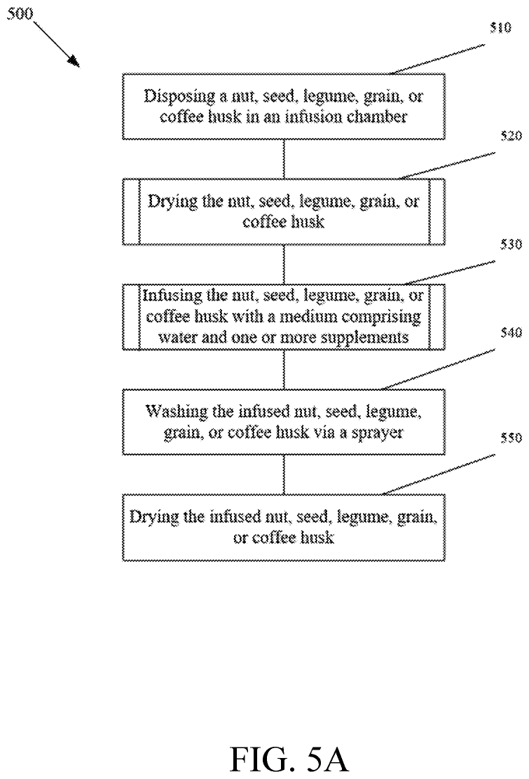

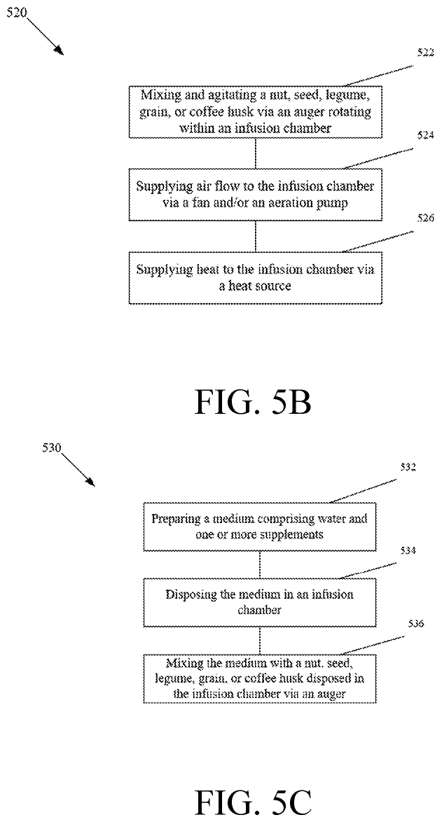

As shown in FIG. 5A, the system 300 or the system 400 can be used in a method 500 for infusing nuts, seeds, legumes, grains or coffee husks. The method 500 may start at block 510, wherein a nut, seed, legume, grain or coffee husk is disposed in an infusion chamber. At block 520, the method includes drying the nut, seed, legume, grain, or coffee husk.

The details of drying the nut, seed, legume, grain, or coffee husk are illustrated in FIG. 5B. At block 522, an auger rotates within the infusion chamber, thereby mixing and agitating the nut, seed, legume, grain, or coffee husk. At block 524, a fan and/or an aeration pump supplies air flow to the infusion chamber. In some embodiments, drying includes adding air or other gasses to the nut, seed, legume, grain, or coffee husk. For example, drying methods may include drying by evaporation using methods known to those of skill in the art. For example, air drying may include use of a fan or blower to circulate air around the nut, seed, legume, grain, or coffee husk. In some embodiments, drying includes removing air or other gasses from the nut, seed, legume, grain, or coffee husk. For example, drying methods may include drying in vacuum using methods known to those of skill in the art. At block 526, a heat source (e.g., infrared heater) supplies heat to the infusion chamber. In some embodiments, drying includes adding heat to the nut, seed, legume, grain, or coffee husk. For example, the nut, seed, legume, grain or coffee husk may be heated according to methods known to those of skill in the art. In some embodiments, drying includes subtracting heat from the nut, seed, legume, grain, or coffee husk. For example, the nut, seed, legume, grain, or coffee husk may be freeze-dried according to methods known to those of skill in the art. In some embodiments, the nut, seed, legume, grain, or coffee husk is dried within the infusion chamber. In some embodiments, the nut, seed, legume, grain, or coffee husk is dried before being added to the infusion chamber, for example using fans, heat, freeze-drying, or another method known to those of skill in the art, and the dried nut, set, legume, grain, or coffee husk is added to the infusion chamber.

In some embodiments, the nut, seed, legume, grain or coffee husk is dried to an equilibrium relative humidity (ERH) of about 0.1% to 60% ERH. For example, the nut, seed, legume, grain or coffee husk may be dried to an equilibrium relative humidity (ERH) of about 0.1%, about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60% ERH, or any value therebetween, or a range constructed from any of the aforementioned values. In some embodiments, the nut, seed, legume, grain or coffee husk is dried to an equilibrium relative humidity of about 0.1% to 15% ERH. In some embodiments, the nut, seed, legume, grain or coffee husk is dried to an equilibrium relative humidity of about 5% to 15% ERH. In some embodiments, the nut, seed, legume, grain or coffee husk is dried to an equilibrium relative humidity of about 8% to 10% ERH. In some embodiments, seeds (including coffee beans) are dried to an ERH of about 8 to about 10%. In some embodiments, nuts are dried to an ERH of about 9 to about 10%.

In some embodiments, the nut, seed, legume, grain or coffee husk is dried according to methods known to those of skill in the art to achieve a desired relative humidity of the nut, seed, legume, grain or coffee husk. For example, methods of drying may include sun drying, air drying, oven drying, dehydrator, freeze drying, microwave drying, spray drying, vacuum drying, drum drying, belt drying, smoke drying, radiant energy drying, or a combination of any of these. For example, one of skill in the art may select the parameters of time and intensity of drying depending on the product to be made and the raw material used. For example, coffee seeds (coffee beans) may be dried from a starting ERH of 12%. Nuts and other seeds may be dried from a similar starting ERH.

In some embodiments, the nut, seed, legume, grain or coffee husk is dried for a period of 1 to 24 hours. In some embodiments, the nut, seed, legume, grain or coffee husk to is dried for a period of about 5 minutes, about 10 minutes, about 20 minutes, about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, or a value therebetween, or range constructed from any of the aforementioned values. In some embodiments, the nut, seed, legume, grain or coffee husk is dried for a period of about 4 to about 6 hours.

### Infusing the Nut, Seed, Legume, Grain or Coffee Husk

As shown in FIG. 5A, the method 500 may proceed to process block 530, wherein the nut, seed, legume, grain or coffee husk is infused with a medium comprising water, and one or more supplements. FIG. 5C is a block diagram that illustrates the details on the methods taking place within the process block 530, wherein the nut, seed, legume, grain or coffee husk is infused with a medium comprising water, and one or more supplements. In some embodiments the one or more supplements include a collagen, collagen derivative, collagen booster, or collagen alternative. At block 532, the method includes preparing a medium comprising water and one or more supplements. The medium can be prepared internally within the system or externally to the system, e.g. by mixing, boiling, agitating, or other methods as disclosed elsewhere herein. At block 534, the method includes disposing the medium in an infusion chamber. At block 536, the method includes mixing the medium with the nut, seed, legume, grain, or coffee husk disposed in the infusion chamber via the auger. At step 538, the method includes allowing the nut, seed, legume, grain or coffee husk to absorb the medium. In some embodiments, the medium may be recirculated from the bottom to the top of a container, for example by a sprayer which collects medium at the bottom of a container and redistributes the medium on top of the nut, seed, legume, grain or coffee husk.

### Washing the Infused Nut, Seed, Legume, Grain or Coffee Husk

As shown in FIG. 5A, the method 500 may proceed to block 540, wherein the infused nut, seed, legume, grain or coffee husk is washed via a sprayer.

For example, washing may be performed to remove residual media dried on the outside of the nut, seed, legume, grain or coffee husk. For example, washing may be performed in a resource-efficient and/or time efficient manner according to methods known to those of skill in the art. For example, in some embodiments the infused nut, seed, legume, grain or coffee husk is washed with water, such as purified water.

The infused nut, seed, legume, grain or coffee husk may be washed using methods known to those of skill in the art. For example, the infused nut, seed, legume, grain or electrostatic washing, chemical washing, enzymatic washing, ozone washing, ionized water washing, high-pressure washing, vibration washing, or a combination of any of these.

### Drying the Infused Nut, Seed, Legume, Grain or Coffee Husk

As shown in FIG. 5A, the method 500 may proceed to block 550, wherein the infused nut, seed, legume, grain or coffee husk is dried.

The infused nut, seed, legume, grain or coffee husk may be dried using methods known to those of skill in the art. For example, in some embodiments the infused nut, seed, legume, grain or coffee husk is dried using any of the methods described with respect to block 520 and FIG. 5B, including a combination of these methods. In some embodiments, drying is staged to not overly heat and reduce quality of the final product. For example, heat may be increased as humidity of the drying nut, seed, legume, grain or coffee husk lowers.

In some embodiments, the infused nut, seed, legume, grain or coffee husk is dried according to methods known to those of skill in the art to achieve a desired relative humidity of the nut, seed, legume, grain or coffee husk. For example, one of skill in the art may select the parameters of time and intensity of drying depending on the product to be made and the raw material used. For example, the final desired relative humidity may depend on the raw material used (for example nuts or seeds, coffee, cascara coffee, almonds, peanuts, etc.). For example, one of skill in the art may select methods of drying the infused nut, seed, legume, grain or coffee husk in order to obtain a product suitable for human consumption and/or organoleptic preservation. In some embodiments, the infused nut, seed, legume, grain or coffee husk is dried within the infusion chamber. In some embodiments, the nut, seed, legume, grain, or coffee husk is dried after being removed from the infusion chamber.

In some embodiments, the infused nut, seed, legume, grain or coffee husk is dried to an equilibrium relative humidity (ERH) of about 0.1% to 60% ERH. For example, the infused nut, seed, legume, grain or coffee husk may be dried to an equilibrium relative humidity (ERH) of about 0.1%, about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60% ERH, or any value therebetween, or a range constructed from any of the aforementioned values. In some embodiments, the infused nut, seed, legume, grain or coffee husk is dried to an equilibrium relative humidity (ERH) of about 0.1% to about 20%. In some embodiments, the infused nut, seed, legume, grain or coffee husk is dried to an equilibrium relative humidity (ERH) of about 5% to about 20%. In some embodiments, the infused nut, seed, legume, grain or coffee husk is dried to an equilibrium relative humidity (ERH) of about 8% to about 12%. In some embodiments, an infused coffee bean is dried to about 12% ERH. In some embodiments, an infused nut (such as an almond) is dried to about 6% ERH. In some embodiments, an infused legume (such as a peanut) is dried to about 7.5% ERH. In some embodiments, an infused coffee husk is dried to about 8% ERH.

In some embodiments, the infused nut, seed, legume, grain or coffee husk to is allowed to dry for a period of about 5 minutes, about 10 minutes, about 20 minutes, about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours, about 51 hours, about 52 hours, about 53 hours, about 54 hours, about 55 hours, about 56 hours, about 57 hours, about 58 hours, about 59 hours, about 60 hours, about 61 hours, about 62 hours, about 63 hours, about 64 hours, about 65 hours, about 66 hours, about 67 hours, about 68 hours, about 69 hours, about 70 hours, or a value therebetween, or range constructed from any of the aforementioned values. In some embodiments, the infused nut, seed, legume, grain or coffee husk to is allowed to dry for no more than 60 hours.

### EXAMPLES

Some aspects of the embodiments discussed above are disclosed in further detail in the following examples, which are not in any way intended to limit the scope of the present disclosure. Those in the art will appreciate that many other embodiments also fall within the scope of the disclosure, as it is described herein above and in the claims.

### Example 1

In the following example, coffee beans were infused with granular HCP.

Pre-drying: Coffee beans were pre-dried from 12% ERH to the ERH indicated in Table 3 below using fans and heat.

Preparation of Infusion Medium: The infusion medium was prepared with water, raw product (here, coffee beans) and one or more supplements. First, the raw product was weighed and the infusion medium ingredients (water, and one or more supplements) were calculated and measured according to the amounts listed in the table below. To prepare the infusion medium, 10-20% of the total water was added to a container, then the one or more supplements were added to the container and mixed. Finally, the remaining water was added while mixing for the time listed in the table below.

Infusion: Once the infusion medium was prepared, the raw product was added and mixed vigorously with the medium in order to achieve a good consistency and homogeneity. During infusion, the mixture was stirred according to the time intervals listed in the table below. The raw product was allowed to absorb the infusion medium according to the time parameters listed in the table below.

TABLE 3

| | | | | | | Medium | Infusion | | Kjeldahl |
|---|---|---|---|---|---|---|---|---|---|
| | | | | Pre-Dry | Filtered | Mixing | Stirring | Infusion | Protein |
| | Raw | Amount | | Humidity | Water | Frequency | Frequency | Time | Content |
| Sample | Product | (g) | Supplement(s) | (% ERH) | (g) | (min) | (min) | (hr) | (% g/g) |
| 1 | Coffee bean | 300 | HCP (bovine), 75 g | 12.0 | 277.251 | 15 | 30 | 8-10 | 23.88% |

Washing: Once the infusion time had elapsed, the collagen and binding supplements on the outside of the grains were removed by washing with water.

Final drying: The infused product was dried to reach an equilibrium relative humidity (% ERH) of between 10-12.5% ERH. The infused product was stirred 3 times a day minimum and was subjected to heat and fan according to Table 4 below:

TABLE 4

| Equilibrium Relative | |
|---|---|
| Humidity (% ERH) | Fan/Heat |
| Above 20% (coffee beans | Fan at 100% without fire |
| look and feel wet) | |
| 20-15% | Fan at 100% and low heat |
| 15-11.5% | Fan at 100% and high heat |

Once the drying process is finished, the fire was turned off and the fan was left on for 10-20 minutes until the infused coffee beans were cooled down.

Results: Infused coffee beans were analyzed for protein content. As noted in Table 3, the infused coffee beans included 23.88% protein content, compared to 10 to 13% in natural coffee beans.

### Example 2

In the following example, nuts, seeds (such as coffee beans), legumes, grains and coffee husks were infused with one or more supplements using the method described in Example 1 and according to the parameters listed in Table 5 below.

Samples 2-4, 6-7, and 9-12 used granular HCP. Sample 5 used powdered HCP. After the infusion of Sample 5, coffee beans were left sticky and adhered to one another after final drying.

TABLE 5

| | | | | | | Medium | Infusion | |
|---|---|---|---|---|---|---|---|---|
| | | | | Pre-Dry | | Mixing | Stirring | Infusion |
| | Raw | Amount | | Humidity | Filtered | Frequency | Frequency | Time |
| Sample | Product | (g) | Supplement(s) | (% ERH) | Water (g) | (min) | (min) | (hr) |
| 2 | Coffee bean | 100 | HCP (bovine), 10 g; Biotin, 0.09 g | 10.0 | 69.91 | 15 | 15 | 4-6 |
| 3 | Coffee bean | 100 | HCP (bovine), 10 g; Biotin, 0.09 g; | 10.0 | 65.753 | 15-45 | 15 | 4-6 |
| | | | Other Supplements* | | | | | |
| 4 | Coffee bean | 100 | HCP (bovine), 28 g; Biotin, 0.09 g | 8.5 | 66.91 | 15 | 30 | 8-10 |
| 5 | Coffee bean | 100 | HCP (bovine), 15 g | 12.0 | 55.45 | 15 | 15 | 6-8 |
| 6 | Coffee bean | 100 | HCP (bovine), 28 g | 12.0 | 70.976 | 15 | 30 | 8-10 |
| 7 | Coffee bean | 100 | HCP (bovine), 30 g | 10.0 | 50 | 15 | 30 | 8-10 |
| 8 | Coffee bean | 100 | Magnesium, 2.55 g; Zinc, 0.18 g | 8.5 | 92.27 | 15-30 | 30 | 4-6 |
| 9 | Coffee bean | 100 | HCP (bovine), 35 g | 9.0 | 55 | 15 | 30 | 10-12 |
| 10 | Peanut | 100 | HCP (bovine), 35 g | 9.0 | 55 | 15 | 30 | 10-12 |
| 11 | Lentil | 100 | HCP (bovine), 35 g | 9.0 | 55 | 15 | 30 | 10-12 |
| 12 | Coffee | 50 | HCP (bovine), 17.5 g | 9.0 | 27.5 | 15 | 30 | 10-12 |
| | cascara | | | | | | | |

*Niacin (0.074 g), Niacinamide (0.074 g), L-arginine (1.98 g), L-Citrulline (0.049 g), L-carnitine (0.198 g), L-Tyrosine (0.891 g), and Acetal L-Tyrosine (NALT) (0.891 g).

### Example 3

In the following example, raw green coffee beans were infused with one or more supplements, as further described below and as shown in Ex. 1 of Table 6. The coffee beans had a density of approximately 673 kg/m³.

### Method

Step 1: Pre-Drying: The green coffee beans were not pre-dried.
Step 2: Solution: Solute (collagen protein) was mixed with solvent (filtered water) according to solubility logic, adding heat or pressure if needed (on average about 15 minutes per solute). Solvent/solute mixing time was approximately 30 minutes.
Step 3: Infusion: Green coffee beans were submerged in the mixture of solvent and solute. Infusion was performed by a cellular respiratory process (with oxygen present, directly or indirectly) for 8 hours with stirring every 15 minutes at a temperature of about 20° C. and a wind flow of about 1500 CFM.
Step 4: Wash: Washing was done as needed to clean residual solution. If the solution had a potential negative flavor, texture effect and/or roasting effect, more washing was performed. The infused objects were washed for approximately 5 minutes.
Step 5: Drying: Drying was staged by quality standards. 20%+ Humidity: Fan Only (20-25° C.), 20% to 15% Humidity: Fan and light heat (30-35° C.); 15% to Standard Humidity: Fan and high heat (40-45° C.). Infused coffee beans were dried to approximately 11.5% relative humidity. Drying was completed over approximately 2.5 days.
Step 6: Dried infused coffee beans were roasted for approximately 20 minutes and brewed for extract for approximately 5 minutes.

TABLE 6

| | | | | | Solution per 100 [symbol] | | | | |
|---|---|---|---|---|---|---|---|---|---|
| | | | | | | Solution | | | Inf Ratio |
| | | | | | | Ratio [symbol] | | | [symbol] Part |
| | | | Obj_Solute [symbol] | Solute | Amount | Solute [symbol] | Total | Object | Object [symbol] |
| [symbol] | EX | Type | %_Solvent | (Supplement) | in Grams | Solvent | Solution | Humidity [symbol] | Solution |
| [symbol] Coffee | 1 | [symbol] | [symbol] _ [symbol] _W | [symbol] Protein- | 70 | 3.50 | 90 | [symbol] | 90.00% |
| Roasted | | | | [symbol] | | | | | |
| Ground & | 2 | [symbol] | [symbol] _ [symbol] _W | [symbol] Protein- | 71.5 | 3.25 | 93.5 | [symbol] | [symbol] |
| [symbol] | | | | [symbol] | | | | | |
| [symbol] ensity: [symbol] | 3 | [symbol] | [symbol] _ [symbol] _W | [symbol] Protein- | [symbol] | 2.20 | [symbol] | [symbol] | 96.00% |
| | | | | [symbol] | | | | | |
| | 4 | [symbol] | [symbol] _ [symbol] _W | [symbol] Protein- | [symbol] | 2.30 | [symbol] | [symbol] | 92.40% |
| | | | | [symbol] | | | | | |
| | 5 | [symbol] | [symbol] _ [symbol] _W | [symbol] Protein- | 72 | 2.00 | 108 | −3 | 108.00% |
| | | | | [symbol] | | | | | |
| | 6 | Extract Per | | | N/A | N/A | N/A | N/A | N/A |
| | | Cup | | | | | | | |
| | 7 | [symbol] | [symbol] _MAG [symbol] | Mineral Zinc | 15 | 15.00 | 16 | −3 | [symbol] |
| | | | ZINC [symbol] _W | Mineral Magnesium | [symbol] | 25.00 | [symbol] | −3 | 65.00% |
| | 8 | Extract Per | | Mineral Zinc | N/A | N/A | N/A | N/A | N/A |
| | | Cup | | Mineral Magnesium | | | | | |
| [symbol] | 9 | Green (Raw) [symbol] | NPEA_ [symbol] _W | [symbol] Protein- | 72 | 2.00 | 108 | −3 | [symbol] |
| [symbol] eanuts [symbol] | | Whole | | [symbol] | | | | | |
| [symbol] ensity: [symbol] | 10 | Green (Raw) [symbol] | NPEA_MAG [symbol] | Mineral Zinc | 15 | 15.00 | 16 | −3 | 16.00% |
| | | Whole | ZINC [symbol] _W | Mineral Magnesium | [symbol] | 25.00 | 65 | −3 | 65.00% |
| [symbol] | 11 | Green (Raw) [symbol] | LLEN_ [symbol] _W | [symbol] Protein- | 72 | 2.00 | [symbol] | −3 | 108.00% |
| [symbol] | | Whole | | [symbol] | | | | | |
| [symbol] ensity: [symbol] | 12 | Green (Raw) [symbol] | LLEN_MAG [symbol] | Mineral Zinc | 15 | 15.00 | 16 | −3 | 16.00% |
| | | Whole | ZINC [symbol] _W | Mineral Magnesium | 62.5 | 25.00 | 65 | −3 | 65.00% |
| Grains - Rice | 13 | Green (Raw) [symbol] | [symbol] _ [symbol] _W | [symbol] Protein- | 72 | 2.00 | 108 | −3 | 108.00% |
| [symbol] ensity: [symbol] | | Whole | | [symbol] | | | | | |
| [symbol] | 14 | Green (Raw) [symbol] | [symbol] _MAG [symbol] | Mineral Zinc | 15 | 15.00 | 16 | −3 | 16.00% |
| | | Whole | ZINC [symbol] _W | Mineral Magnesium | 62.5 | 25.00 | 65 | −3 | 65.00% |
| [symbol] - Coffee | 15 | Green (Raw) [symbol] | [symbol] _ [symbol] _W | [symbol] Protein- | 72 | 2.00 | [symbol] | −3 | 108.00% |
| [symbol] | | Whole | | [symbol] | | | | | |
| [symbol] | 16 | Green (Raw) [symbol] | [symbol] _MAG [symbol] | Mineral Zinc | 15 | 15.00 | 16 | −3 | 16.00% |
| [symbol] ensity: [symbol] | | Whole | ZINC [symbol] _W | Mineral Magnesium | [symbol] | 25.00 | 65 | −3 | 65.00% |

| | | | Added G | | | | | | Solute | |
|---|---|---|---|---|---|---|---|---|---|---|
| | | Nat G | per 100 G | Results | | | Grams | | (Sup) | |
| | | per 100 | [symbol] or | G per | Increase | Infusion | per 16.67 | Extract | Soluble | Solute |
| [symbol] | EX | G [symbol] | Extract | 100 G | by X | % | Serving | Rate [symbol] | Rate | Class |
| [symbol] Coffee | 1 | 8 | 20 | [symbol] | 2.40 | 56% | 3.20 | N/A | 1:2-4 | Very |
| Roasted | 2 | 8 | 22 | 21.22 | [symbol] | 60% | 3.54 | N/A | 1:2-4 | Very |
| Ground & | 3 | 8 | 30 | [symbol] | 2.99 | 53% | 3.98 | N/A | 1:2-4 | Very |
| [symbol] | 4 | 8 | 28 | 23.01 | 2.88 | 54% | 3.84 | N/A | 1:2-4 | Very |
| [symbol] ensity: [symbol] | 5 | 8 | 36 | 20.23 | 2.53 | 34% | 3.37 | N/A | 1:2-4 | Very |
| | 6 | 0.35 | N/A | 1.42 | 4.06 | | 1.42 | 42% | | Very |
| | 7 | 0.00035 | 1 | 0.00099 | 2.83 | 0.1% | 0.00 | N/A | [symbol] | More |
| | | 0.003 | 2.5 | [symbol] | 63.77 | 8% | 0.03 | N/A | [symbol] | Slightly |
| | 8 | 0.00002 | 1 | 0.00004 | 2.00 | 0.002% | 0.00004 | 24% | [symbol] | More |
| | | 0.0003 | 2.5 | 0.00121 | 4.03 | 0.04% | 0.00121 | 4% | [symbol] | Slightly |
| [symbol] | 9 | [symbol] | [symbol] | 31.62 | 1.23 | 16% | 5.27 | N/A | 1:2-4 | Very |
| [symbol] eanuts [symbol] | 10 | 0.00037 | 1 | 0.12762 | 344.92 | [symbol] | 0.02 | N/A | [symbol] | More |
| [symbol] ensity: | | [symbol] | 2.5 | [symbol] | 1.40 | 3% | 0.04 | N/A | [symbol] | Slightly |
| [symbol] | | | | | | | | | | |
| [symbol] | 11 | 15.11 | 36 | 29.74 | 1.97 | 41% | 4.96 | N/A | 1:2-4 | Very |
| [symbol] | 12 | 0.00478 | 1 | 0.24586 | 51.44 | 24% | 0.04 | N/A | [symbol] | More |
| [symbol] ensity: [symbol] | | 0.122 | 2.5 | 0.13532 | 1.11 | 1% | 0.02 | N/A | [symbol] | Slightly |
| Grains - Rice | 13 | 4.3 | 36 | 9.55 | 2.22 | 15% | 1.59 | N/A | 1:2-4 | Very |
| [symbol] ensity: [symbol] | 14 | 0.00213 | 1 | 0.0888 | 41.69 | 9% | 0.01 | N/A | [symbol] | More |
| [symbol] | | 0.012 | 2.5 | 0.3171 | 26.43 | 12% | 0.05 | N/A | [symbol] | Slightly |
| [symbol] - Coffee | 15 | 12.5 | 36 | 23.01 | 1.84 | 29% | 0.83 | N/A | 1:2-4 | Very |
| [symbol] | 16 | 0.0004 | 1 | [symbol] | 38.15 | 1.5% | 0.00 | N/A | [symbol] | More |
| [symbol] | | 0.025 | 2.5 | [symbol] | [symbol] | [symbol] | 0.01 | N/A | [symbol] | Slightly |
| [symbol] ensity: [symbol] | | | | | | | | | | |

[symbol]

indicates data missing or illegible when filed

### Example 4

In the following example, raw green coffee beans were infused with one or more supplements, as further described below and as shown in Ex. 2 of Table 6. The coffee beans had a density of approximately 673 kg/m³.

### Method

Step 1: Pre-Drying: The green coffee beans were not pre-dried.
Step 2: Solution: Solute (collagen protein) was mixed with solvent (filtered water) according to solubility logic, adding heat or pressure if needed (on average about 15 minutes per solute). Solvent/solute mixing time was approximately 30 minutes.
Step 3: Infusion: Green coffee beans were submerged in the mixture of solvent and solute. Infusion was performed by a cellular respiratory process (with oxygen present, directly or indirectly) for 8 hours with stirring every 15 minutes at a temperature of about 20° C. and a wind flow of about 1500 CFM.
Step 4: Wash: Washing was done as needed to clean residual solution. If the solution had a potential negative flavor, texture effect and/or roasting effect, more washing was performed. The infused objects were washed for approximately 5 minutes.
Step 5: Drying: Drying was staged by quality standards. 20%+ Humidity: Fan Only (20-25° C.), 20% to 15% Humidity: Fan and light heat (30-35° C.); 15% to Standard Humidity: Fan and high heat (40-45° C.). Infused coffee beans were dried to approximately 11.5% relative humidity. Drying was completed over approximately 2.5 days.
Step 6: Dried infused coffee beans were roasted for approximately 20 minutes and brewed for extract for approximately 5 minutes.

### Example 5

In the following example, raw green coffee beans were infused with one or more supplements, as further described below and as shown in Ex. 3 of Table 6. The coffee beans had a density of approximately 673 kg/m³.

### Method

Step 1: Pre-Drying: The green coffee beans were not pre-dried.
Step 2: Solution: Solute (collagen protein) was mixed with solvent (filtered water) according to solubility logic, adding heat or pressure if needed (on average about 15 minutes per solute). Solvent/solute mixing time was approximately 30 minutes.
Step 3: Infusion: Green coffee beans were submerged in the mixture of solvent and solute. Infusion was performed by a cellular respiratory process (with oxygen present, directly or indirectly) for 8 hours with stirring every 15 minutes at a temperature of about 20° C. and a wind flow of about 1500 CFM.
Step 4: Wash: Washing was done as needed to clean residual solution. If the solution had a potential negative flavor, texture effect and/or roasting effect, more washing was performed. The infused objects were washed for approximately 5 minutes.
Step 5: Drying: Drying was staged by quality standards. 20%+ Humidity: Fan Only (20-25° C.), 20% to 15% Humidity: Fan and light heat (30-35° C.); 15% to Standard Humidity: Fan and high heat (40-45° C.). Infused coffee beans were dried to approximately 11.5% relative humidity. Drying was completed over approximately 2.5 days.
Step 6: Dried infused coffee beans were roasted for approximately 20 minutes and brewed for extract for approximately 5 minutes.

### Example 6

In the following example, raw green coffee beans were infused with one or more supplements, as further described below and as shown in Ex. 4 of Table 6. The coffee beans had a density of approximately 673 kg/m³.

### Method

Step 1: Pre-Drying: The green coffee beans were not pre-dried.
Step 2: Solution: Solute (collagen protein) was mixed with solvent (filtered water) according to solubility logic, adding heat or pressure if needed (on average about 15 minutes per solute). Solvent/solute mixing time was approximately 30 minutes.
Step 3: Infusion: Green coffee beans were submerged in the mixture of solvent and solute. Infusion was performed by a cellular respiratory process (with oxygen present, directly or indirectly) for 8 hours with stirring every 15 minutes at a temperature of about 20° C. and a wind flow of about 1500 CFM.
Step 4: Wash: Washing was done as needed to clean residual solution. If the solution had a potential negative flavor, texture effect and/or roasting effect, more washing was performed. The infused objects were washed for approximately 5 minutes.
Step 5: Drying: Drying was staged by quality standards. 20%+ Humidity: Fan Only (20-25° C.), 20% to 15% Humidity: Fan and light heat (30-35° C.); 15% to Standard Humidity: Fan and high heat (40-45° C.). Infused coffee beans were dried to approximately 11.5% relative humidity. Drying was completed over approximately 2.5 days.
Step 6: Dried infused coffee beans were roasted for approximately 20 minutes and brewed for extract for approximately 5 minutes.

### Example 7

In the following example, raw green coffee beans were infused with one or more supplements, as further described below and as shown in Ex. 5-6 of Table 6. The coffee beans had a density of approximately 673 kg/m³.

### Method

Step 1: Pre-Drying: Green coffee beans were pre-dried as needed for maximum infusion to reduce standard object humidity by minus 1% to minus 4% (On average 1 hour of drying per minus 1%). Less density correlated to greater volume capacity. Object was pre-dried for approximately 3 hours.
Step 2: Solution: Solute (collagen protein) was mixed with solvent (filtered water) according to solubility logic, adding heat or pressure if needed (on average about 15 minutes per solute). Solvent/solute mixing time was approximately 30 minutes.
Step 3: Infusion: Green coffee beans were submerged in the mixture of solvent and solute. Infusion was performed by a cellular respiratory process (with oxygen present, directly or indirectly) for 8 hours with stirring every 15 minutes at a temperature of about 20° C. and a wind flow of about 1500 CFM.
Step 4: Wash: Washing was done as needed to clean residual solution. If the solution had a potential negative flavor, texture effect and/or roasting effect, more washing was performed. The infused objects were washed for approximately 5 minutes.
Step 5: Drying: Drying was staged by quality standards. 20%+ Humidity: Fan Only (20-25° C.), 20% to 15% Humidity: Fan and light heat (30-35° C.); 15% to Standard Humidity: Fan and high heat (40-45° C.). Infused coffee beans were dried to approximately 11.5% relative humidity. Drying was completed over approximately 2.5 days.
Step 6: Dried infused coffee beans were roasted for approximately 20 minutes and brewed for extract for approximately 5 minutes.

### Example 8

In the following example, raw green coffee beans were infused with one or more supplements, as further described below and as shown in Ex. 7-8 of Table 6. The coffee beans had a density of approximately 673 kg/m³.

### Method

Step 1: Pre-Drying: Green coffee beans were pre-dried as needed for maximum infusion to reduce standard object humidity by minus 1% to minus 4% (On average 1 hour of drying per minus 1%). Less density correlated to greater volume capacity. Object was pre-dried for approximately 3 hours.
Step 2: Solution: Solute (mineral zinc, mineral magnesium) was mixed with solvent (filtered water) according to solubility logic, adding heat or pressure if needed (on average about 15 minutes per solute). Solvent/solute mixing time was approximately 30 minutes.
Step 3: Infusion: Green coffee beans were submerged in the mixture of solvent and solute. Infusion was performed by a cellular respiratory process (with oxygen present, directly or indirectly) for 8 hours with stirring every 15 minutes at a temperature of about 20° C. and a wind flow of about 1500 CFM.
Step 4: Wash: Washing was done as needed to clean residual solution. If the solution had a potential negative flavor, texture effect and/or roasting effect, more washing was performed. The infused objects were washed for approximately 5 minutes.
Step 5: Drying: Drying was staged by quality standards. 20%+ Humidity: Fan Only (20-25° C.), 20% to 15% Humidity: Fan and light heat (30-35° C.); 15% to Standard Humidity: Fan and high heat (40-45° C.). Infused coffee beans were dried to approximately 11.5% relative humidity. Drying was completed over approximately 2.5 days.
Step 6: Dried infused coffee beans were roasted for approximately 20 minutes and brewed for extract for approximately 5 minutes.

### Example 9

In the following example, peanuts were infused with one or more supplements, as further described below and as shown in Ex. 9 of Table 6. The peanuts had a density of approximately 617 kg/m³.

### Method

Step 1: Pre-Drying: Peanuts were pre-dried as needed for maximum infusion to reduce standard object humidity by minus 1% to minus 4% (On average 1 hour of drying per minus 1%). Less density correlated to greater volume capacity. Object was pre-dried for approximately 3 hours.
Step 2: Solution: Solute (collagen protein) was mixed with solvent (filtered water) according to solubility logic, adding heat or pressure if needed (on average about 15 minutes per solute). Solvent/solute mixing time was approximately 30 minutes.
Step 3: Infusion: Peanuts were submerged in the mixture of solvent and solute. Infusion was performed by a cellular respiratory process (with oxygen present, directly or indirectly) for 8 hours with stirring every 15 minutes at a temperature of about 20° C. and a wind flow of about 1500 CFM.
Step 4: Wash: Washing was done as needed to clean residual solution. If the solution had a potential negative flavor, texture effect and/or roasting effect, more washing was performed. The infused objects were washed for approximately 5 minutes.
Step 5: Drying: Drying was staged by quality standards. 20%+ Humidity: Fan Only (20-25° C.), 20% to 15% Humidity: Fan and light heat (30-35° C.); 15% to Standard Humidity: Fan and high heat (40-45° C.). Peanuts were dried to approximately 7.5% relative humidity. Drying was completed over approximately 2.5 days.

### Example 10

In the following example, peanuts were infused with one or more supplements, as further described below and as shown in Ex. 10 of Table 6. The peanuts had a density of approximately 617 kg/m³.

### Method

Step 1: Pre-Drying: Peanuts were pre-dried as needed for maximum infusion to reduce standard object humidity by minus 1% to minus 4% (On average 1 hour of drying per minus 1%). Less density correlated to greater volume capacity. Object was pre-dried for approximately 3 hours.
Step 2: Solution: Solute (mineral zinc, mineral magnesium) was mixed with solvent (filtered water) according to solubility logic, adding heat or pressure if needed (on average about 15 minutes per solute). Solvent/solute mixing time was approximately 30 minutes.
Step 3: Infusion: Peanuts were submerged in the mixture of solvent and solute. Infusion was performed by a cellular respiratory process (with oxygen present, directly or indirectly) for 8 hours with stirring every 15 minutes at a temperature of about 20° C. and a wind flow of about 1500 CFM.
Step 4: Wash: Washing was done as needed to clean residual solution. If the solution had a potential negative flavor, texture effect and/or roasting effect, more washing was performed. The infused objects were washed for approximately 5 minutes.
Step 5: Drying: Drying was staged by quality standards. 20%+ Humidity: Fan Only (20-25° C.), 20% to 15% Humidity: Fan and light heat (30-35° C.); 15% to Standard Humidity: Fan and high heat (40-45° C.). Peanuts were dried to approximately 7.5% relative humidity. Drying was completed over approximately 2.5 days.

### Example 11

In the following example, lentils were infused with one or more supplements, as further described below and as shown in Ex. 11 of Table 6. The lentils had a density of approximately 811 kg/m³.

### Method

Step 1: Pre-Drying: Lentils were pre-dried as needed for maximum infusion to reduce standard object humidity by minus 1% to minus 4% (On average 1 hour of drying per minus 1%). Less density correlated to greater volume capacity. Object was pre-dried for approximately 3 hours.
Step 2: Solution: Solute (collagen protein) was mixed with solvent (filtered water) according to solubility logic, adding heat or pressure if needed (on average about 15 minutes per solute). Solvent/solute mixing time was approximately 30 minutes.
Step 3: Infusion: Lentils were submerged in the mixture of solvent and solute. Infusion was performed by a cellular respiratory process (with oxygen present, directly or indirectly) for 8 hours with stirring every 15 minutes at a temperature of about 20° C. and a wind flow of about 1500 CFM.
Step 4: Wash: Washing was done as needed to clean residual solution. If the solution had a potential negative flavor, texture effect and/or roasting effect, more washing was performed. The infused objects were washed for approximately 5 minutes.
Step 5: Drying: Drying was staged by quality standards. 20%+ Humidity: Fan Only (20-25° C.), 20% to 15% Humidity: Fan and light heat (30-35° C.); 15% to Standard Humidity: Fan and high heat (40-45° C.). Lentils were dried to approximately 14% relative humidity. Drying was completed over approximately 2.5 days.

### Example 12

In the following example, lentils were infused with one or more supplements, as further described below and as shown in Ex. 12 of Table 6. The lentils had a density of approximately 811 kg/m³.

### Method

Step 1: Pre-Drying: Lentils were pre-dried as needed for maximum infusion to reduce standard object humidity by minus 1% to minus 4% (On average 1 hour of drying per minus 1%). Less density correlated to greater volume capacity. Object was pre-dried for approximately 3 hours.
Step 2: Solution: Solute (mineral zinc, mineral magnesium) was mixed with solvent (filtered water) according to solubility logic, adding heat or pressure if needed (on average about 15 minutes per solute). Solvent/solute mixing time was approximately 30 minutes.
Step 3: Infusion: Lentils were submerged in the mixture of solvent and solute. Infusion was performed by a cellular respiratory process (with oxygen present, directly or indirectly) for 8 hours with stirring every 15 minutes at a temperature of about 20° C. and a wind flow of about 1500 CFM.
Step 4: Wash: Washing was done as needed to clean residual solution. If the solution had a potential negative flavor, texture effect and/or roasting effect, more washing was performed. The infused objects were washed for approximately 5 minutes.
Step 5: Drying: Drying was staged by quality standards. 20%+ Humidity: Fan Only (20-25° C.), 20% to 15% Humidity: Fan and light heat (30-35° C.); 15% to Standard Humidity: Fan and high heat (40-45° C.). Lentils were dried to approximately 14% relative humidity. Drying was completed over approximately 2.5 days.

### Example 13

In the following example, grains of rice were infused with one or more supplements, as further described below and as shown in Ex. 13 of Table 6. The rice had a density of approximately 845 kg/m³.

### Method

Step 1: Pre-Drying: Raw rice was pre-dried as needed for maximum infusion to reduce standard object humidity by minus 1% to minus 4% (On average 1 hour of drying per minus 1%). Less density correlated to greater volume capacity. Object was pre-dried for approximately 3 hours.
Step 2: Solution: Solute (collagen protein) was mixed with solvent (filtered water) according to solubility logic, adding heat or pressure if needed (on average about 15 minutes per solute). Solvent/solute mixing time was approximately 30 minutes.
Step 3: Infusion: Raw rice was submerged in the mixture of solvent and solute. Infusion was performed by a cellular respiratory process (with oxygen present, directly or indirectly) for 8 hours with stirring every 15 minutes at a temperature of about 20° C. and a wind flow of about 1500 CFM.
Step 4: Wash: Washing was done as needed to clean residual solution. If the solution had a potential negative flavor, texture effect and/or roasting effect, more washing was performed. The infused objects were washed for approximately 5 minutes.
Step 5: Drying: Drying was staged by quality standards. 20%+ Humidity: Fan Only (20-25° C.), 20% to 15% Humidity: Fan and light heat (30-35° C.); 15% to Standard Humidity: Fan and high heat (40-45° C.). Raw rice was dried to approximately 14% relative humidity. Drying was completed over approximately 2.5 days.

### Example 14

In the following example, grains of rice were infused with one or more supplements, as further described below and as shown in Ex. 14 of Table 6. The rice had a density of approximately 845 kg/m³.

### Method

Step 1: Pre-Drying: Raw rice was pre-dried as needed for maximum infusion to reduce standard object humidity by minus 1% to minus 4% (On average 1 hour of drying per minus 1%). Less density correlated to greater volume capacity. Object was pre-dried for approximately 3 hours.
Step 2: Solution: Solute (mineral zinc, mineral magnesium) was mixed with solvent (filtered water) according to solubility logic, adding heat or pressure if needed (on average about 15 minutes per solute). Solvent/solute mixing time was approximately 30 minutes.
Step 3: Infusion: Raw rice was submerged in the mixture of solvent and solute. Infusion was performed by a cellular respiratory process (with oxygen present, directly or indirectly) for 8 hours with stirring every 15 minutes at a temperature of about 20° C. and a wind flow of about 1500 CFM.
Step 4: Wash: Washing was done as needed to clean residual solution. If the solution had a potential negative flavor, texture effect and/or roasting effect, more washing was performed. The infused objects were washed for approximately 5 minutes.
Step 5: Drying: Drying was staged by quality standards. 20%+ Humidity: Fan Only (20-25° C.), 20% to 15% Humidity: Fan and light heat (30-35° C.); 15% to Standard Humidity: Fan and high heat (40-45° C.). Raw rice was dried to approximately 14% relative humidity. Drying was completed over approximately 2.5 days.

### Example 15

In the following example, coffee husks were infused with one or more supplements, as further described below and as shown in Ex. 15 of Table 6. The coffee husks had a density of approximately 982 kg/m³.

### Method

Step 1: Pre-Drying: Coffee husks were pre-dried as needed for maximum infusion to reduce standard object humidity by minus 1% to minus 4% (On average 1 hour of drying per minus 1%). Less density correlated to greater volume capacity. Object was pre-dried for approximately 3 hours.
Step 2: Solution: Solute (collagen protein) was mixed with solvent (filtered water) according to solubility logic, adding heat or pressure if needed (on average about 15 minutes per solute). Solvent/solute mixing time was approximately 30 minutes.
Step 3: Infusion: Coffee husks were submerged in the mixture of solvent and solute. Infusion was performed by a cellular respiratory process (with oxygen present, directly or indirectly) for 8 hours with stirring every 15 minutes at a temperature of about 20° C. and a wind flow of about 1500 CFM.
Step 4: Wash: Washing was done as needed to clean residual solution. If the solution had a potential negative flavor, texture effect and/or roasting effect, more washing was performed. The infused objects were washed for approximately 5 minutes.
Step 5: Drying: Drying was staged by quality standards. 20%+ Humidity: Fan Only (20-25° C.), 20% to 15% Humidity: Fan and light heat (30-35° C.); 15% to Standard Humidity: Fan and high heat (40-45° C.). Coffee husks were dried to approximately 8% relative humidity. Drying was completed over approximately 2.5 days.

### Example 16

In the following example, coffee husks were infused with one or more supplements, as further described below and as shown in Ex. 16 of Table 6. The coffee husks had a density of approximately 982 kg/m³.

### Method

Step 1: Pre-Drying: Coffee husks were pre-dried as needed for maximum infusion to reduce standard object humidity by minus 1% to minus 4% (On average 1 hour of drying per minus 1%). Less density correlated to greater volume capacity. Object was pre-dried for approximately 3 hours.
Step 2: Solution: Solute (mineral zinc, mineral magnesium) was mixed with solvent (filtered water) according to solubility logic, adding heat or pressure if needed (on average about 15 minutes per solute). Solvent/solute mixing time was approximately 30 minutes.
Step 3: Infusion: Coffee husks were submerged in the mixture of solvent and solute. Infusion was performed by a cellular respiratory process (with oxygen present, directly or indirectly) for 8 hours with stirring every 15 minutes at a temperature of about 20° C. and a wind flow of about 1500 CFM.
Step 4: Wash: Washing was done as needed to clean residual solution. If the solution had a potential negative flavor, texture effect and/or roasting effect, more washing was performed. The infused objects were washed for approximately 5 minutes.
Step 5: Drying: Drying was staged by quality standards. 20%+ Humidity: Fan Only (20-25° C.), 20% to 15% Humidity: Fan and light heat (30-35° C.); 15% to Standard Humidity: Fan and high heat (40-45° C.). Coffee husks were dried to approximately 8% relative humidity. Drying was completed over approximately 2.5 days.

### Example 17

Further infusions were performed according to Table 7 below. Green coffee beans were pre-dried. A liquid medium was created by mixing solute (listed in Table 7) with water. Green coffee beans were infused with the liquid medium, washed, and dried post infusion.

TABLE 7

| Stage | Element | Process: Manual | | | | | | | | |
|---|---|---|---|---|---|---|---|---|---|---|
| Object: | Seed (Coffee) | All Coffee Types, Limited Nuts (Arabica, | | | | | | | | |
| | | Robusta; Caffeinated, Decaffeinated) | | | | | | | | |
| | Std Humidity | 12% | | | | | | | | |
| | Pre-Dry Process Humidity | 8.5% | | | | | | | | |
| | Level | | | | | | | | | |
| | High Heat (Celsius) | 45-50 | | | | | | | | |
| | | C. | | | | | | | | |
| | Time (Hours) | 4 Hours | | | | | | | | |
| | Serving Size (Grams) | TBD | | | | | | | | |
| Solution: | Solvent: Water or Oil Based | Filtered Water | | | | | | | | |
| Solvent + | Object: Solution [symbol] Ratio % | 100% | 100% | 100% | 100% | 110% | 120% | 80% | 65% | 65% |
| [symbol] | (Object Weight [symbol] | | | | | | | | | |
| | Solution % [symbol] | | | | | | | | | |
| | Solute(s) [symbol] | | | | | | | | | |
| | Solvent amount) | | | | | | | | | |
| | Solute: Supplement(s) - (e.g. | Amino Acids | | | | Gen Health | | Caffeine | | Mushrooms |
| | the most soluble version | | | | | | | | | |
| | available) | L-Arginine | L- [symbol] | L-Tyrosine | L-Tyrosine | Collagen | Creatine | Guarana | [symbol] | Lions |
| | | | | | (NALT) | (Bovine) | | | | Mane |
| | Amount Per Serving (Grams) | 500 | 500 | 500 | 0.35 | 2 | 2 | 0.675 | 0.200 | 500 |
| | Fusion Process Loss × Factor | 2 | | | | | | | | |
| | Fusion [symbol] Per Serving (G) | 1000 | 1000 | 1000 | 0.7 | 4 | 4 | 1.35 | 0.4 | 1000 |
| | Type: (EXP - Extract Powder; | EX | EX | EX | NAP | GN | MPD | PD | EX | EX |
| | GN - Granular; PD - Powder; | | | | | | | | | |
| | MPD - Micro Powder; NAP - N- | | | | | | | | | |
| | Acetyl Powder) | | | | | | | | | |
| | Solubility Factor (1-5) | 4 | 4 | 4 | [symbol] | [symbol] | 2 | [symbol] | [symbol] | [symbol] |
| | Stirring (Mins) | 10 | 10 | 10 | 10 | 10 | 10 | 10 | 10 | 10 |
| | Heat (Celsius) | 60 | 60 | 60 | 60 | 80 | 100 | 60 | 60 | 60 |
| | Cooling (Mins) | 10 | 10 | 10 | 10 | 15 | 20 | 10 | 10 | 10 |
| Fusion: | Time (Hours) | 6 | 6 | 6 | 6 | 10 | 12 | 6 | 6 | 6 |

| | | |
|---|---|---|
| Object + | Airflow | Open Ltd. |
| Solution | Drying | N/A |
| | Cycling Speed (Bottom to Top) | N/A |
| | Heat | Environmental Limited |
| | Oxidation | Open Limited |
| | Consistency | Each Batch Different |
| | Results | Clumpy, Average 10-20% Supplements [symbol] |
| [symbol] | Wash Cycles | 3 |
| Post- | Dry Time (Hours) | 12 |
| Drying | Airflow (RPM) | Max |
| | [symbol] | Low (20-25): Humidity 20%+ |
| | | [symbol] |

| Stage | Element | Process: Manual | | | | | | | | |
|---|---|---|---|---|---|---|---|---|---|---|
| Object: | Seed (Coffee) | All Coffee Types, Limited Nuts (Arabica, | | | | | | | | |
| | | Robusta; Caffeinated, Decaffeinated) | | | | | | | | |
| | Std Humidity | 12% | | | | | | | | |
| | Pre-Dry Process Humidity | 8.5% | | | | | | | | |
| | Level | | | | | | | | | |
| | High Heat (Celsius) | 45-50 | | | | | | | | |
| | | C. | | | | | | | | |
| | Time (Hours) | 4 Hours | | | | | | | | |
| | Serving Size (Grams) | TBD | | | | | | | | |
| Solution: | Solvent: Water or Oil Based | Filtered Water | | | | | | | | |
| Solvent + | Object: Solution [symbol] Ratio % | 65% | 65% | 65% | 65% | 100% | 65% | 65% | 100% | 100% |
| [symbol] | (Object Weight [symbol] | | | | | | | | | |
| | Solution % [symbol] | | | | | | | | | |
| | Solute(s) [symbol] | | | | | | | | | |
| | Solvent amount) | | | | | | | | | |
| | Solute: Supplement(s) - (e.g. | Mushrooms | | | | Vit/Min | | | | |
| | the most soluble version | | | | | | | | | |
| | available) | Chage | Reishi | Cordyceps | Others | Biotin | Niacin | Niacinamide | Zinc | Magnesium |
| | Amount Per Serving (Grams) | 500 | 500 | 500 | 500 | 0.05 | 0.15 | 0.15 | 2 | 0.4 |
| | Fusion Process Loss × Factor | 2 | | | | | | | | |
| | Fusion [symbol] Per Serving (G) | 1000 | 1000 | 1000 | 1000 | 0.1 | 0.3 | 0.3 | 4 | [symbol] |
| | Type: (EXP - Extract Powder; | EX [symbol] | EX [symbol] | EX [symbol] | EX | EX | EX | EX | EX | EX |
| | GN - Granular; PD - Powder; | | | | | | | | | |
| | MPD - Micro Powder; NAP - N- | | | | | | | | | |
| | Acetyl Powder) | | | | | | | | | |
| | Solubility Factor (1-5) | [symbol] | [symbol] | [symbol] | [symbol] | [symbol] | [symbol] | [symbol] | 2 | 2 |
| | Stirring (Mins) | 10 | 10 | 10 | 10 | 10 | 10 | 10 | 10 | 10 |
| | Heat (Celsius) | 60 | 60 | 60 | 60 | 60 | 60 | 60 | 100 | 100 |
| | Cooling (Mins) | 10 | 10 | 10 | 10 | 10 | 10 | 10 | 20 | 20 |
| Fusion: | Time (Hours) | 6 | 6 | 6 | 6 | 6 | 6 | 6 | 12 | 12 |

| | | |
|---|---|---|
| Object + | Airflow | Open Ltd. |
| Solution | Drying | N/A |
| | Cycling Speed (Bottom to Top) | N/A |
| | Heat | Environmental Limited |
| | Oxidation | Open Limited |
| | Consistency | Each Batch Different |
| | Results | Clumpy, Average 10-20% Supplements [symbol] |
| [symbol] | Wash Cycles | 3 |
| Post- | Dry Time (Hours) | 12 |
| Drying | Airflow (RPM) | Max |
| | [symbol] | Low (20-25): Humidity 20%+ |
| | | [symbol] |

[symbol]

indicates data missing or illegible when filed

### Example 18

Further infusions were performed using the method described in FIGS. 5A-5C and the system shown in FIGS. 3A-3C and 4A-4C, according to Table 8 below.

Green coffee beans were added to the infusion chamber. The green coffee beans were pre-dried to an ERH of 8.5%. Green coffee beans were infused with a medium comprising water, and one or more supplements in the infusion chamber. The infused green coffee beans were washed and dried in the infusion chamber.

TABLE 8

| Stage | Element | Process: Machine | | | | | | | |
|---|---|---|---|---|---|---|---|---|---|
| Object: | Seed (Coffee) | All Coffee Types, Limited Nuts (Arabica, | | | | | | | |
| | | Robusta; Caffeinated, Decaffeinated) | | | | | | | |
| | Std Humidity | 12% | | | | | | | |
| | Pre-Dry Process Humidity | 8.5% | | | | | | | |
| | Level | | | | | | | | |
| | High Heat (Celsius) | 45-50 | | | | | | | |
| | | C. | | | | | | | |
| | Time (Hours) | 4 Hours | | | | | | | |
| | Serving Size (Grams) | TBD | | | | | | | |
| Solution: | Solvent: Water or Oil Based | Filtered Water | | | | | | | |
| Solvent + | Object: Solution − Ratio % | 100% | 100% | 100% | 100% | 110% | 120% | 65% | 65% |
| Solute | (Object Weight * Solution % − | | | | | | | | |
| | Solute(s) = Solvent amount) | | | | | | | | |
| | Solute: Supplement(s) - (e.g. | Amino Acids | | | | Gen Health | | Caffeine | |
| | the most soluble version | | | | | | | | |
| | available) | L-Arginine | L-Citrulline | L-Tyrosine | L-Tyrosine | Collagen | Creatine | Guarana | Synthetic |
| | | | | | (NALT) | (Bovine) | | | |
| | Amount Per Serving (Grams) | 0.375 | 0.375 | 0.375 | 0.3 | 1.7 | 1.7 | 0.575 | 0.17 |
| | Fusion Process Loss × Factor | 1.75 | | | | | | | |
| | Fusion Amt Per Serving (G) | 0.7 | 0.7 | 0.7 | 0.5 | 3.0 | 3.0 | 1.0 | 0.3 |
| | Type: (EXP - Extract Powder; | EX | EX | EX | NAP | GN | MPD | PD | EX |
| | GN - Granular; PD - Powder; | | | | | | | | |
| | MPD - Micro Powder; NAP - N- | | | | | | | | |
| | Acetyl Powder) | | | | | | | | |
| | Solubility Factor (1-5) | 5 | 5 | 5 | 5 | 3 | 2 | 5 | 5 |
| | Stirring (Mins) | #REF! | 10 | 10 | 10 | 10 | 10 | 10 | 10 |
| | Heat (Celsius) | 60 | 60 | 60 | 60 | 80 | 100 | 60 | 60 |
| | Cooling (Mins) | 10 | 10 | 10 | 10 | 15 | 20 | 10 | 10 |
| Fusion: | Time (Hours) | 3 | 3 | 3 | 3 | 6 | 8 | 3 | 3 |
| Object + | Airflow | MED | MED | MED | MED | HIGH | HIGH | LOW | LOW |
| Solution | Drying | | | | | | | | |
| | Cycling Speed (Bottom to Top) | | | | | | | | |
| | Heat | | | | | | | | |
| | Oxidation | | | | | | | | |

| | | |
|---|---|---|
| | Consistency | Homogeneous, Estimated Less than 1-2% Variance |
| | Results | No Clumping, Only 3-7% Estimated Waste |
| Wash + | Wash Cycles | 3 |
| Post- | Dry Time (Hours) | 12 |
| Drying | Airflow (RPM) | Max |
| | Heat (Celsius) | Low (20-25): Humidity 20%+ |
| | | Medium (30-35): Humidity 20-15% |
| | | High (40-45): Humidity 15-12% |

| Stage | Element | Process: Machine | | | | | | | | | |
|---|---|---|---|---|---|---|---|---|---|---|---|
| Object: | Seed (Coffee) | All Coffee Types, Limited Nuts (Arabica, | | | | | | | | | |
| | | Robusta; Caffeinated, Decaffeinated) | | | | | | | | | |
| | Std Humidity | 12% | | | | | | | | | |
| | Pre-Dry Process Humidity | 8.5% | | | | | | | | | |
| | Level | | | | | | | | | | |
| | High Heat (Celsius) | 45-50 | | | | | | | | | |
| | | C. | | | | | | | | | |
| | Time (Hours) | 4 Hours | | | | | | | | | |
| | Serving Size (Grams) | TBD | | | | | | | | | |
| Solution: | Solvent: Water or Oil Based | Filtered Water | | | | | | | | | |
| Solvent + | Object: Solution − Ratio % | 65% | 65% | 65% | 65% | 65% | 65% | 65% | 65% | 120% | 120% |
| Solute | (Object Weight * Solution % − | | | | | | | | | | |
| | Solute(s) = Solvent amount) | | | | | | | | | | |
| | Solute: Supplement(s) - (e.g. | Mushrooms | | | | | Vit/Min | | | | |
| | the most soluble version | | | | | | | | | | |
| | available) | Lions | Chage | Reishi | Cordyceps | Others | Biotin | Niacin | Niacinamide | Zinc | Magnesium |
| | | Mane | | | | | | | | | |
| | Amount Per Serving (Grams) | 0.375 | 0.375 | 0.375 | 0.375 | 0.375 | 0.035 | 0.170 | 0.170 | 2 | 0.36 |
| | Fusion Process Loss × Factor | 1.75 | | | | | | | | | |
| | Fusion Amt Per Serving (G) | 0.7 | 0.7 | 0.7 | 0.7 | 0.7 | 0.1 | 0.3 | 0.3 | 3.5 | 0.6 |
| | Type: (EXP - Extract Powder; | EX | EX | EX | EX | EX | EX | EX | EX | EX | EX |
| | GN - Granular; PD - Powder; | | | | | | | | | | |
| | MPD - Micro Powder; NAP - N- | | | | | | | | | | |
| | Acetyl Powder) | | | | | | | | | | |
| | Solubility Factor (1-5) | 5 | 5 | 5 | 5 | 5 | 5 | 5 | 5 | 2 | 2 |
| | Stirring (Mins) | 10 | 10 | 10 | 10 | 10 | 10 | 10 | 10 | 10 | 10 |
| | Heat (Celsius) | 60 | 60 | 60 | 60 | 60 | 60 | 60 | 60 | 100 | 100 |
| | Cooling (Mins) | 10 | 10 | 10 | 10 | 10 | 10 | 10 | 10 | 20 | 20 |
| Fusion: | Time (Hours) | 3 | 3 | 3 | 3 | 3 | 3 | 3 | 3 | 8 | 8 |
| Object + | Airflow | LOW | LOW | LOW | LOW | LOW | MED | LOW | LOW | MED | MED |
| Solution | Drying | | | | | | | | | | |
| | Cycling Speed (Bottom to Top) | | | | | | | | | | |
| | Heat | | | | | | | | | | |
| | Oxidation | | | | | | | | | | |

| | | |
|---|---|---|
| | Consistency | Homogeneous, Estimated Less than 1-2% Variance |
| | Results | No Clumping, Only 3-7% Estimated Waste |
| Wash + | Wash Cycles | 3 |
| Post- | Dry Time (Hours) | 12 |
| Drying | Airflow (RPM) | Max |
| | Heat (Celsius) | Low (20-25): Humidity 20%+ |
| | | Medium (30-35): Humidity 20-15% |
| | | High (40-45): Humidity 15-12% |

### Example 19

In the following example, the caffeine content of guarana infused coffee produced using the method described in Example 17 and Table 7 was compared to the caffeine content of normal (non-infused) coffee. Both coffees were made (40 mL) from the same batch of starting coffee source and amounts. The guarana infused coffee had caffeine content of 173.83 mg per 100 mL while normal coffee had a caffeine content of 99.11 mg per 100 mL.

### Example 20

The results of a guarana coffee bean infusion using a manual process and using a machine were compared. The results are shown in Tables 9 and 10 below.

TABLE 9

| Process: Manual | | Grams/Status |
|---|---|---|
| Object: UGQ SPC Colombian Coffee | | 5000 |
| Solution 80% | | 4000 |
| Solute: Guarana Supp Natural Powder | | 300 |
| Solute: Guarana Supp Total | | 300 |
| Solvent: Water | | 3700 |
| Elements: | Time | 6 Hours |
| | Movement (Food Drill Bit Drilling) | Random Limited |
| | Cycling (Man Pull while drilling) | Random Limited |
| | Airflow (Open Air) | Open Limited |
| | Heat (At Environment Level) | Environmental Limited |
| | Oxidation/Gas (Open Air) | Open Limited |
| Results: | Consistency | Random Limited |
| | Color | Spotty |
| | Capacity | Basic |
| | Quality | Good |
| | Lab Verified - Caffeine | 0.173 |

TABLE 10

| Process: Machine | | Grams/Status |
|---|---|---|
| Object: UGQ SPC Colombian Coffee | | 5000 |
| Solution 80% | | 4000 |
| Solute: Guarana Supp Natural Powder | | 398 |
| Solute: Guarana Supp Total | | 398 |
| Solvent: Water | | 3603 |
| Elements: | Time | 2.25 Hours |
| | Movement (Auger) | Low Managed |
| | Cycling (Auger) | Low Mngd |
| | Airflow (Set Speed Fan) | Low Mngd |
| | Heat (Set Temp Control) | Mngd 32 C. |
| | Oxidation/Gas (Bottom to Top thru Fusion) | As Needed |
| Results: | Consistency | Total |
| | Color | Uniform |
| | Capacity | Max |
| | Quality | Great |
| | Lab Verified - Caffeine | 0.200 |

### Other Considerations

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” “involving,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y or Z,” unless specifically stated otherwise, is otherwise understood with the context as used in general to present that an item, term, etc., may be either X, Y or Z, or any combination thereof (such as X, Y and/or Z). Thus, such disjunctive language is not generally intended to, and should not, imply that certain embodiments require at least one of X, at least one of Y or at least one of Z to each be present.

The terms “about” or “approximate” and the like are synonymous and are used to indicate that the value modified by the term has an understood range associated with it, where the range can be ±20%, ±15%, ±10%, ±5%, or ±1%. The term “substantially” is used to indicate that a result (such as a measurement value) is close to a targeted value, where close can mean, for example, the result is within 80% of the value, within 90% of the value, within 95% of the value, or within 99% of the value.

Unless otherwise explicitly stated, articles such as “a” or “an” should generally be interpreted to include one or more described items.

While the above detailed description has shown, described, and pointed out novel features as applied to illustrative embodiments, it will be understood that various omissions, substitutions, and changes in the form and details of the devices or algorithms illustrated can be made without departing from the spirit of the disclosure. As will be recognized, certain embodiments described herein can be embodied within a form that does not provide all of the features and benefits set forth herein, as some features can be used or practiced separately from others. All changes which come within the meaning and range of equivalency of the claims are to be embraced within their scope.

It should be appreciated that all combinations of the foregoing concepts (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

The scope of the present disclosure is not intended to be limited by the specific disclosures of examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A method to fortify nuts, seeds, legumes, grains or coffee husks with one or more supplements, the method comprising:
drying a nut, seed, legume, grain or coffee husk to an equilibrium relative humidity (ERH) of about 0.1% to 60% ERH; and
infusing the nut, seed, legume, grain or coffee husk with a medium comprising water, and one or more supplements, the one or more supplements comprising collagen, a collagen derivative, a collagen booster, a collagen alternative, a mushroom or a combination thereof.

2. The method of claim 1, wherein the one or more supplements comprise a collagen derivative.

3. The method of claim 2, wherein the collagen derivative comprises granulated hydrolyzed collagen peptides.

4. The method of claim 1, wherein the one or more supplements comprise collagen.

5. The method of claim 1, wherein the one or more supplements comprise a collagen booster.

6. The method of claim 1, wherein the one or more supplements comprise a collagen alternative.

7. The method of claim 1, wherein the ERH is about 0.1% to 15% ERH.

8. The method of claim 1, wherein infusing comprises mixing the medium and the nut, seed, legume, grain or coffee husk by recirculating the medium, pumping the medium, or agitating a mixture of the medium and the nut, seed, legume, grain or coffee husk.

9. The method of claim 1, wherein infusing comprises allowing the nut, seed, legume, grain or coffee husk to absorb the medium for a period of 10 minutes to 24 hours.

10. The method of claim 1, wherein the medium further comprises a surfactant, enzyme, or emulsifier.

11. The method of claim 1, wherein drying comprises adding heat to or subtracting heat from the nut, seed, legume, grain or coffee husk.

12. The method of claim 1, wherein drying comprises adding air or other gasses to or subtracting air or other gasses from the nut, seed, legume, grain or coffee husk.

13. The method of claim 1, wherein the nut, seed, legume, grain or coffee husk is left to infuse until a desired level of infusion or absorption is achieved.

14. The method of claim 1, wherein the collagen, collagen derivative, collagen booster, collagen alternative, or combination thereof comprises gelatin, hydrolyzed collagen peptides, pure collagen, partially pure collagen, or a combination thereof.

15. A method to infuse nuts, seeds, legumes, grains or coffee husks with one or more supplements, the method comprising:
(a) drying the nuts, seeds, legumes, grains or coffee husks to an equilibrium relative humidity (ERH) of about 0.1% to 15% ERH;
(b) mixing the nuts, seeds, legumes, grains or coffee husks and a medium comprising water and one or more supplements;
(c) allowing the nuts, seeds, legumes, grains or coffee husks to absorb the medium, thereby infusing the nuts, seeds, legumes, grains or coffee husks;
(d) washing the infused nuts, seeds, legumes, grains or coffee husks with water; and
(e) drying the infused nuts, seeds, legumes, grains or coffee husks to 0.1-20% ERH.

16. A system for infusing granular solids, the system comprising:
a tank comprising:
an opening configured to receive granular solids and one or more liquids;
an infusion chamber in fluidic communication with the opening;
a liquid drain in fluidic communication with the infusion chamber; and
an outlet in fluidic communication with the infusion chamber;
an air source in fluidic communication with the infusion chamber;
a plurality of air channels positioned on the length of the infusion chamber, wherein the plurality of air channels connects the air source to the infusion chamber; and
an auger positioned interior to the infusion chamber.

17. A method to fortify nuts, seeds, legumes, grains or coffee husks with one or more supplements, the method comprising:
(a) disposing a nut, seed, legume, grain, or coffee husk in an infusion chamber;
(b) drying a nut, seed, legume, grain or coffee husk to an equilibrium relative humidity (ERH) of about 0.1% to 60% ERH;
(c) infusing the nut, seed, legume, grain, or coffee husk with a medium comprising water, and one or more supplements in the infusion chamber; and
(d) drying the infused nut, seed, legume, grain, or coffee husk in the infusion chamber.
